(19) United States
(12) Patent Application Publication
(10) Pub. No.: US 2003/0049699 A1
(43) Pub. Date: Mar. 13, 2003

(54) Polypeptide (MBP1) capable of interacting with oncogenic mutants of the P53 protein

(76) Inventors: Emmanuel Conseiller, Paris (FR); Laurent Debussche, Athis-Mons (FR); William Gallagher, Paris (FR)

Correspondence Address: WILEY, REIN & FIELDING, LLP, ATTN: PATENT ADMINISTRATION, 1776 K. STREET N.W., WASHINGTON, DC, 20006, US

(21) Appl. No.: 09/829,936

(22) Filed: Apr. 11, 2001

Related U.S. Application Data
(60) Provisional application No. 60/132,331, filed on May 3, 1999.

(30) Foreign Application Priority Data
Oct. 12, 1999 (US) PCT/FR99/02465
Oct. 12, 1998 (FR) 98/12754

(51) Int. Cl. G01N 33/574

(52) U.S. Cl. 435/7.23

(57) ABSTRACT

The invention concerns the field of biology and cell cycle regulation. More particularly, the invention concerns novel polypeptides capable of interacting specifically with an oncogenic form of the p53 protein.

The present invention relates to the field of biology and regulation of the cell cycle. More particularly, the present invention relates to new polypeptides capable of interacting specifically with the oncogenic forms of the p53 protein

The wild-type p53 protein is involved in the regulation of the cell cycle and in maintaining the integrity of the cell genome. This protein, whose main function is being an activator of the transcription of certain genes, is capable of blocking the cell in the G1 phase of the cell cycle during the appearance of mutations during the replication of the genome, and of triggering a number of DNA repair processes. This blocking in the G1 phase is due mainly to the activation of the p21/WAF1 gene. Furthermore, in the event of poor functioning of these repair processes and in the event of the appearance of mutational events which are too numerous to be corrected, this protein is capable of inducing the phenomenon of programmed cell death, called apoptosis.

In this manner, the p53 protein acts as a tumour suppressor, by eliminating abnormally differentiated cells and cells whose genome has been damaged.

The p53 protein contains 393 amino acids, which define 5 functional domains (see FIG. 1):

the transcription activating domain, consisting of amino acids 1 to 73, which is capable of binding certain factors for the general transcription machinery such as the TEP protein. This domain is also the seat of a number of post-translational modifications. It is also the seat of numerous interactions of the p53 protein with numerous other proteins and in particular with the MDM2 cellular protein or the EBNA5 protein of the Epstein-Barr virus (EBV), which are capable of blocking the function of the wild-type protein. Furthermore, this domain possesses so-called PEST amino acid sequences for susceptibility to proteolytic degradation.

the DNA-binding domain, located between amino acids 73 and 315. The conformation of this central domain of p53 regulates the recognition of DNA sequences specific for the p53 protein. This domain is the seat of two types of alterations affecting the function of the wild-type protein:

(i) the interaction with proteins blocking the function of the p53 protein such as the “large T” antigen of the SV40 virus or the E6 viral proteins of the HPV16 and HPV18 viruses capable of causing its degradation by the ubiquitin system. The latter interaction may occur in the presence of the cellular protein E6ap (E3 enzyme of the ubiquitinilation cascade).

(ii) the point mutations which affect the function of the p53 protein and of which practically all that have been observed in human cancers are located in this region,

the nuclear localization signal, consisting of amino acids 315 to 325, which is essential for proper addressing of the protein in the compartment where it will exert its main function.

the oligomerization domain, consisting of amino acids 325 to 355. This 325 to 355 region forms a structure of the type: β sheet (326-334)-bend (335-336)-α helix (337-355). The alterations of functions located in this region are essentially due to the interaction of the wild type protein with the various mutant forms which may lead to variable effects on the function of the wild-type protein.

the regulatory domain, consisting of amino acids 365 to 393, which is the seat of a number of post-translational modifications (glycosylations, phosphorylations, binding of RNA, and the like) which modulate the function of the p53 protein in a positive or negative manner. This domain plays an extremely important role in the modulation of the activity of the wild-type protein.

The function of the p53 protein may be disrupted in various ways:

by blocking of its function by a number of factors such as for example the “large T” antigen of the SV40 virus, the EBNA5 protein of the Epstein-Barr virus, or the MDM2 cellular protein,

by destabilization of the protein by increasing its susceptibility to proteolysis, in particular by interaction with the E6 protein of the human papillomavirus HPV16 and HPV18, which promotes the entry of p53 into the ubiquitinilation cycle. In this case, the interaction between these two proteins can only occur through the prior binding of a cellular protein, the E6ap protein whose binding site is poorly known,

by point mutations at the level of the p53 protein gene,

by deletion of one or two p53 alleles

The last two types of modifications are found in about 50% of the various types of cancer. In this regard, the mutations in the p53 protein gene recorded in cancer cells affect a very large portion of the gene encoding this protein, and result in variable modifications of the function of this protein. It may be noted, however, that the great majority of these mutations are located in the central part of the p53 protein which is known to be the region of contact with the genomic sequences specific for the p53 protein. This explains why the main characteristic of most of the mutations in the p53 protein is that of no longer being able to bind to the DNA sequences which the wild-type protein recognizes and thus of no longer being able to exert their role as transcription factor.

All these modifications are currently grouped into two categories:

the so-called weak mutants, whose product is a nonfunctional protein, which, in the case of a mutation on only one of the two alleles, does not affect the function of the wild-type protein encoded by the other allele. The main representative of this category is the H273 mutant specific for the Li-Fraumeni familial syndrome for hypersensitivity to cancer conditions,

the dominant-oncogenic mutants, whose product is a protein which has lost the capacity to bind to DNA and which actively participates in the neoplastic transformation. The mutants of this category have lost their transactivating capacity and are more stable than the wild-type protein. They are incapable of inhibiting the transformation of rat embryonic fibroblasts and their function as oncogenes by cooperating with the activated form of RAS in the transformation of rat embryonic fibroblasts (Eliyahu et al, Nature 312 (1984) 646/Parada et al, Nature 312 (1984) 649). This behaviour may be explained by two different mechanisms which are mutually nonexclusive;

(i) these mutants generate a nonfunctional protein, which, in the case of a mutation on only one of the two alleles and through interaction with the wild-type protein, is capable of blocking its function by the formation of nonactive mixed oligomers which can no longer bind to the DNA sequences specific for the wild-type protein. Such a mechanism is invoked in the case where malignant transformation of the cells is observed after transfection of the mutants in the presence of endogenous p53.

(ii) these mutants may, furthermore, exhibit a “gain of function” phenotype. Their expression in nontumorigenic cells not expressing endogenous p53 leads to the appearance of tumours in athymic mice (Dittmer et al, Nature Genetics 4 (1993) 42). These mutants are capable of activating the transcription of genes such as MDR or PCNA which do not have consensus sequences recognized by p53, which activation probably occurs through the recruitment of transcription factors specific for the mutants and which may participate in the appearance of the tumour phenotype (Chin et al, Science 255 (1992) 459; Deb et al, J. Virol. 66 (1992) 6164). Finally, it has recently been reported that these mutants can disrupt the attachment of certain regions of DNA (MAR/SAR) to the nuclear matrix network (Muller et al, Oncogene 12 (1996) 1941).

Many cellular partners have been described for the p53 protein. Some interact both with the wild-type and mutated conformations of the protein and others as specific for one or other of the conformations (Iwabuchi et al, Proc. Natl. Acad. Sci. USA 91 (1994) 6098). It is conceivable that some of these “gain or function” properties may be mediated by protein partners specific for the p53 mutants; however, such partners have so far never been identified. The identification of such partners would make possible new approaches in anticancer therapies based on the modification or the control of these interactions and on the production of compounds capable of interfering with the interaction of these protein partners with the different forms of p53. The present invention satisfies this need and provides, in addition, other advantages.

With the aim of studying this “gain or function” phenotype capable of involving protein-protein interactions specific for this type of mutant, the double-hybrid system was used to search for partners specific for the H175 mutant, the main representative of this category of mutants. A mouse embryo cDNA library, fused with the sequence of the transactivating domain of GAL4 (TA), was screened in the yeast strain YCM17 using, as bait protein, the 73-393 domain of the H175 mutant fused with the DNA binding domain of Gal4 (DB). This screening made it possible to isolate two cDNAs encoding two different proteins: the MBP1 protein and Fibulin-2.

The interactions between these two proteins and the H175 mutant of the p53 protein were able to be confirmed in mammalian cells and functional effects were able to be demonstrated, both on the properties of the mutated form of p53 and on the properties of the wild-type form.

The present invention therefore results from the identification, by the applicant, of new polypeptides capable of interacting specifically with various forms of the p53 protein. More precisely, the present invention results from the identification, isolation and characterization of a new protein and of the corresponding gene, said protein being characterized in that it is capable of interacting specifically with the oncogenic forms of p53 and with the mutants H175 and G281 in particular. This protein is called MBP1 for p53 Mutant Binding Protein. The present invention also results from the demonstration that another protein, fibulin 2, is capable of interacting specifically with the oncogenic forms of p53 and with the mutants H175 and G281 in particular.

The present invention also results from the discovery of the particular properties of these new protein partners of p53 which, unexpectedly, are also found to be capable of blocking the antiproliferative effects of the wild-type form of p53.

These new protein partners of p53 exhibit, in addition, a very high synergy of action with the oncogenic mutants of p53, this synergy is exerted both for the oncogenic cooperation with the activated form of the Ras protein and on the proliferative effect of the mutated forms of p53.

Furthermore, and independently of any interaction with p53, these polypeptides exhibit a positive effect on cell growth in addition, one of these partners, the MBP1 protein, exhibits the characteristics of an immortalizing oncogene by cooperating with the activated form of the Ras protein for cellular transformation.

By virtue of the specificity and the synergistic effects which these new partners of p53 exhibit toward certain mutated forms of p53, these polypeptides constitute a therapeutic target of choice for the treatment of cancers linked to mutations in the p53 protein.

In addition, these polypeptides, which exhibit intrinsic oncogenic properties, constitute new potential targets for the treatment of cancer in general.

A first subject of the invention therefore relates to polypeptides capable of interacting specifically with the oncogenic forms of p53. These polypeptides are, in addition, capable of stimulating cell growth and of blocking the antiproliferative effects of the wild-type form of p53.

According to a first embodiment, these polypeptides comprise all or part of a sequence chosen from the polypeptide sequences SEQ ID No. 9 (murine MBP1 C-terminal fragment) or SEQ ID No. 16 (murine MBP1) or a derivative thereof.

According to another embodiment, these polypeptides comprise all or part of a sequence chosen from the polypeptide sequences SEQ ID No. 31 (human MBP1 C-terminal fragement) or SEQ ID No. 22 (human MBP1) or a derivative thereof.

Finally, according to yet another embodiment, these polypeptides comprise all or part of the polypeptide sequence SEQ ID No. 33 (murine Fibulin-2 C-terminal fragment) or a derivative thereof.

Preferably, the polypeptides of the invention are represented by the polypeptide sequence SEQ ID No. 22 or its derivatives.

For the purposes of the present invention, the term polypeptide sequence derivative designates any polypeptide sequence differing from the sequence considered, which is obtained by one or more modifications of a genetic and/or chemical nature, and possessing the capacity to interact with the oncogenic mutated forms of p53. Modification of a genetic and/or chemical nature is understood to mean any mutation, substitution, deletion, addition and/or modification of one or more residues. Such derivatives may be generated for different objectives, such as in particular that of modifying their properties of binding to the oncogenic mutated forms of p53, or of increasing their therapeutic efficacy or of reducing their side effects, or that of conferring on them new pharmacokinetic and/or biological properties.

In this regard, another object of the invention relates to the polypeptide sequences have biological functions comparable to those of the polypeptides according to the invention and in particular the capacity to interact with the oncogenic mutated forms of p53 and which exhibit a degree of identity of at least 80% and preferably of at least 90% with the polypeptide sequence SEQ ID No. 16 or the polypeptide sequence SEQ ID No. 22 or the polypeptide sequence SEQ ID No. 33.

Preferably, the polypeptide sequences according to the invention exhibit at least 95% and still more preferably at least 97% identity with the polypeptide sequence SEQ ID No. 16 or the polypeptide sequence SEQ ID No. 22 or the polypeptide sequence SEQ ID No. 33.

In a more particularly preferred manner, the polypeptide sequences according to the invention exhibit at least 98% identity and still more preferably at least 99% identity with the polypeptide sequence SEQ ID No. 16 or the polypeptide sequence SEQ ID No. 22 or the polypeptide sequence SEQ ID No. 33.

The term polypeptide sequence derivative also comprises the fragments of the polypeptide sequences indicated above. Such fragments may be generated in various ways. In particular, they may be synthesized by the chemical route, on the basis of the sequences given in the present application, using the peptide synthesizers known to persons skilled in the art. They may also be synthesized by the genetic route, by expressing in a cellular host a nucleotide sequence encoding the desired peptide. In this case, the nucleotide sequence may be prepared chemically using an oligonucleotide synthesizer, on the basis of the peptide sequence given in the present application and of the genetic code. The nucleotide sequence may also be prepared from the sequences given in the present application, by enzymatic cleavages, ligation, cloning and the like, according to techniques known to persons skilled in the art, or by screening DNA libraries with probes prepared from these sequences.

Another subject of the present invention relates to the nucleotide sequences SEQ ID No. 15, SEQ ID No. 21 and SEQ ID No. 32, respectively encoding the polypeptide sequences presented in the sequences SEQ ID No. 16 or SEQ ID No. 22 or SEQ ID No. 33.

According to a specific embodiment of the invention, the nucleotide sequences comprise all or part of the sequence SEQ ID No. 15 or SEQ ID No. 21 or their derivatives.

According to another embodiment of the invention, the nucleotide sequences comprise all or part of the nucleotide sequence SEQ ID No. 32 (cDNA corresponding to the murine Fibulin-2 C-term fragment) or its derivatives.

According to yet another embodiment, the nucleotide sequences comprise the sequence SEQ ID No. 23 (murine MBP1 cDNA, partial sequence) or the sequence SEQ ID No. 30 (cDNA corresponding to the human MBP1 C-term fragment.

According to a preferred mode, the nucleotide sequence is represented by the sequence SEQ ID No. 21 or its derivatives.

For the purposes of the present invention, the term nucleotide sequence derivative designates any sequence differing from the sequence considered because of the degeneracy of the genetic code, which is obtained by one or more modifications of a genetic and/or chemical nature, as well any sequence hybridizing with these sequences or fragments thereof and encoding a polypeptide according to the invention. Modification of a genetic and/or chemical nature is understood to mean any mutation, substitution, deletion, addition and/or modification of one or more residues.

The term nucleotide sequence derivative also comprises the sequences homologous to the sequence considered, which are obtained from other cellular sources and in particular from cells of human origin, or from other organisms.

In this regard, the present invention relates to any nucleotide sequence which exhibits at least 70% identity and preferably at least 85% identity with the nucleotide sequence SEQ ID No. 21 or the nucleotide sequence SEQ ID No. 15 or the nucleotide sequence SEQ ID No. 32.

Preferably, the nucleotide sequence according to the invention exhibits at least 90% and still more preferably at least 93% identity with the nucleotide sequence SEQ ID No. 21 or the nucleotide sequence SEQ ID No. 15 or the nucleotide sequence SEQ ID No. 32.

In a more particularly preferred manner, the sequences according to the invention exhibit at least 95% and still more preferably 97%, or even 98% or even 99% identity with the nucleotide sequence SEQ ID No. 21 or the nucleotide sequence SEQ ID No. 15 or the nucleotide sequence SEQ ID No. 32.

Such homologous sequences may be obtained by hybridization experiments. The hybridizations may be carried out using nucleic acid libraries, using as probe the native sequence or a fragment thereof, under varying hybridization conditions.

Another subject of the invention relates to the nucleotide sequences capable of hybridizing under high stringency conditions with the nucleotide sequences defined above.

In this regard, the term high stringency condition means that the hybridization occurs if the nucleotide sequences exhibit at least 95% and preferably at least 97% identity.

As indicated above, such sequences may in particular be used as detection probes which RNA or cDNA or genomic DNA to isolate nucleotide sequences encoding polypeptides according to the invention. Such probes generally have at least 15 bases. Preferably, these probes have at least 30 bases and may have more than 50 bases. Preferably, these probes have been 30 and 50 bases.

The nucleotide sequences according to the invention may be of artificial origin or otherwise. They may be genomic sequences, cDNA, RNA, hybrid sequences or synthetic or semisynthetic sequences. These sequences may be obtained, for example, by screening DNA libraries (cDNA library, genomic DNA library) by means of probes prepared on the basis of sequences presented above. Such libraries may be prepared from cells of various origins by conventional molecular biology techniques known to persons skilled in the art. The nucleotide sequences of the invention may also be prepared by chemical synthesis or alternatively by mixed methods including chemical or enzymatic modification of sequences obtained by screening libraries. In general, the nucleic acids of the invention may be prepared according to any technique known to persons skilled in the art.

For the purposes of the present invention, the name oncogenic forms or oncogenic mutated forms of p53 designates the dominant-oncogenic mutants whose product is a protein which has lost the capacity to bind DNA and which actively participates in the neoplastic transformation. The mutants of this category have lost their transactivating capacity and are more stable than the wild-type protein. The representatives of this category of mutants of p53 are in particular the mutant forms H175, G281, W248 and A143.

Another subject of the present invention relates to a method for preparing the polypeptides according to the invention according to which a cell containing a nucleotide sequence according to the invention is cultured under conditions for expressing said sequence, and the polypeptide produced is recovered. In this case, the part encoding said polypeptide is generally placed under the control of signals allowing its expression in a cellular host. The choice of these signals (promoters, terminators, leader sequence for secretion, and the like) may vary depending on the cellular host used. Moreover, the nucleotide sequences of the invention may form part of a vector which may be autonomously replicating or integrative. More particularly, autonomously-replicating vectors may be prepared using autonomously-replicating sequences in the chosen host. As regards integrative vectors, these may be prepared, for example, using sequences homologous to certain regions of the genome of the host, allowing, by homologous recombination, the integration of the vector.

The subject of the present invention is also host cells transformed with a nucleic acid containing a nucleotide sequence according to the invention. The cellular hosts which can be used for the production of the peptides of the invention by the recombinant route are both eukaryotic and prokaryotic hosts. Among the eukaryotic hosts which are suitable, there may be mentioned animal cells, yeasts or fungi. In particular, as regards yeasts, there may be mentioned yeasts of the genus Saccharomyces, Kluyveromyces, Pichia, Schwanniomyces or Hansenula. As regards animal cells, there may be mentioned insect cells (SF9 or SF21), COS, CHO or C127 cells, human neuroblastomas and the like. Among the fungi, there may be mentioned more particularly Aspergillus ssp. or Trichoderma ssp. As prokaryotic hosts, the use of following bacteria is preferred: E. coli, Bacillus or Streptomyces.

According to a preferred embodiment, the host cells are advantageously represented by recombinant yeast strains for the expression of the nucleic acids of the invention as well as the production of the proteins derived therefrom.

Preferably, the host cells comprise at least a sequence or a fragment of sequence chosen from the nucleotide sequences SEQ ID No. 15, No. 21, No. 32, No. 23 and No. 30 for the production of the polypeptides according to the invention.

Another application of the nucleic acid sequences according to the invention is the production of antisense oligonucleotides or of genetic antisenses which can be used as pharmaceutical agents. The antisense sequences are small-sized oligonucleotides, which are complementary to the coding strand of a given gene, and which are as a result capable of hybridizing specifically with the transcribed mRNA, inhibiting translation into a protein. The subject of the invention is thus the antisense sequences capable of inhibiting, at least partially, the expression of polypeptides capable of interacting with p53 such as the MBP1 protein or fibulin 2. Such sequences may consist of all or part of the nucleotides sequences defined above and may be obtained by fragmentation and the like or by chemical synthesis.

The nucleotide sequences according to the invention may be used for the transfer and production in vitro, in vivo or ex vivo of antisense sequences or for the expression of proteins or polypeptides capable of interacting with the p53 protein.

In this regard, the nucleotide sequences according to the invention may be incorporated into viral or nonviral vectors, allowing their administration in vitro, in vivo or ex vivo.

Another subject of the invention relates, in addition, to any vector comprising a nucleotide sequence defined above. The vector of the invention may be, for example, a plasmid, a cosmid or any DNA not encapsidated by a virus, a phage, an artificial chromosome, a recombinant virus and the like. It is preferably a plasmid or a recombinant virus.

As viral vectors in accordance with the invention, there may be most particularly mentioned vectors of the adenovirus, retrovirus, adeno-associated virus, herpesvirus or vaccinia virus type. The subject of the present application is also defective recombinant viruses comprising a heterologous nucleic sequence encoding a polypeptide according to the invention.

The invention also allows the production of nucleotide probes, synthetic or otherwise, capable of hybridizing with the nucleotide sequences defined above or corresponding mRNAs. Such probes may be used in vitro as a diagnostic tool, for the detection of the polypeptides according to the invention and in particular the human MBP1 protein or fibulin 2. These probes may also be used for the detection of genetic abnormalities (pore splicing, polymorphism, point mutations and the like). These probes may also be used for the detection and isolation of homologous nucleic acid sequences encoding the polypeptides as defined above, from other cellular sources and preferably from cells of human origin. The probes of the invention also contain at least 10 nucleotides, preferably at least 15 nucleotides, and still more preferably at least 20 nucleotides. Preferably, these probes are labelled prior to their use. For that, various techniques known to persons skilled in the art may be used (radioactive or enzymatic labelling and the like).

The invention also relates to the use of nucleotide probes, synthetic or otherwise, capable of hybridizing with nucleotide sequences encoding the MBP1 protein for carrying out diagnostic tests for cancerous tissues based on the detection of the level of expression of MBP1. By way of example of nucleotide probes which can be used for this application, there may be mentioned in particular the sequences SEQ ID No. 27 and SEQ ID No. 28. These nucleotide probes make it possible to detect the amplification of the expression of the MBP1 protein. These probes may be RNA or DNA probes. The present invention demonstrates that an amplification of the messenger RNA encoding the human MBP1 protein maybe detected in some types of human tumours and in particular in the case of colon cancers. In this regard, the invention also relates to a method for the diagnosis of cancer comprising the detection of the amplification of the expression of the gene encoding the human MBP1 protein.

Another subject of the invention consists in polyclonal or monoclonal antibodies or antibody fragments directed against a polypeptide as defined above. Such antibodies may be generated by methods known to persons skilled in the art. In particular, these antibodies may be prepared by immunizing an animal against a polypeptide whose sequence is chosen from the sequences SEQ ID No. 9 (murine MBP1 C-terminal fragment) or SEQ ID No. 31 (human MBP1 C-terminal fragment) or the polypeptide sequences SEQ ID No. 22 (human MBP1) or SEQ ID No. 33 (Fibulin-2 C-term fragment) or any fragment or derivative thereof, and then collecting blood and isolating the antibodies. These antibodies may also be generated by the preparation of hybridomas according to techniques known to persons skilled in the art.

The subject of the invention is also single-chain antibodies ScFv derived from the monoclonal antibodies defined above. Such single-chain antibodies may be obtained according to the techniques described in U.S. Pat. Nos. 4,946,778, 5,132,405 and 5,476,786.

The antibodies or antibody fragments according to the invention may be used in particular for inhibiting and/or revealing the interaction between p53 and the polypeptides as defined above.

Another subject of the present invention relates to a method for identifying compounds capable of binding to the polypeptides according to the invention. The detection and/or isolation of these compounds may be carried out according to the following steps:

a molecule or a mixture containing various molecules, optionally unidentified, is brought into contact with a polypeptide of the invention under conditions allowing interaction between said polypeptide and said molecule in the case where the latter might have affinity for said polypeptide, and,

the molecules bound to said polypeptide of the invention are detected and/or isolated.

According to a specific embodiment, such a method makes it possible to identify molecules capable of preventing or blocking the cell growth stimulating activity of the polypeptides according to the invention and in particular of human MBP1 protein or Fibulin 2 or fragments derived from these proteins. These molecules are also capable of exhibiting anticancer properties and of preventing the immortalizing oncogene function exhibited by MBP1 or the polypeptides derived from MBP1 which cooperate with the activated form of the Ras protein for the cell transformation.

In this regard, another subject of the invention relates to the use of a ligand identified and/or obtained according to the method described above as a medicament. Such ligands are indeed capable of treating certain conditions involving a cell cycle dysfunction and in particular cancers.

Another subject of the present invention relates to a method for identifying compounds capable of modulating or completely or partially inhibiting the interaction between the oncogenic mutated forms of p53 and the polypeptides according to the invention.

The detection and/or isolation of modulators or of ligands capable of modulating or of completely or partially inhibiting the interaction between the oncogenic mutated forms of p53 and the polypeptides according to the invention may be carried out according to the following steps:

a mutated form of p53 or of a fragment thereof is bound to a polypeptide according to the invention; it may be the mutated forms of p53 such as H175, G281, W248 or A143 or a fragment thereof; it is preferably the H175 form or alternatively the G281 form,

a compound to be tested for its capacity to inhibit the binding between the mutated form of p53 and the polypeptides according to the invention is added;

it is determined whether the mutated form of p53 or the polypeptides according to the invention are displaced by the binding or prevented from binding;

the compounds which prevent or which impede the binding between the mutated form of p53 and the polypeptides according to the invention are detected and/or isolated.

In a specific embodiment, this method of the invention is adapted to the detection and/or isolation of agonists or antagonists of the interaction between the mutated forms of p53 and the polypeptides of the invention. Still according to a specific embodiment, the invention provides a method for identifying molecules capable of blocking the interaction between the mutated forms of p53 and the human MBP1 protein or human fibulin 2. Such a method makes it possible to identify molecules capable of preventing the effects of the action of the polypeptides according to the invention with the mutated forms of p53. In particular, such compounds are capable of preventing the oncogenic cooperation between the MBP1 protein and the oncogenic mutant forms of p53 such as in particular H175.

In this regard, another subject of the invention relates to the use of a ligand or a modulator identified and/or obtained according to the method described above as a medicament. Such ligands or modulators are indeed capable of treating certain conditions involving a cell cycle dysfunction and in particular cancers.

The invention also provides nonpeptide or nonexclusively peptide compounds which can be used pharmaceutically. It is indeed possible, using the active protein units described in the present application, to produce molecules inhibiting the interaction of MBP1 or of fibulin2 with the oncogenic mutated forms of p53, these molecules being nonexclusively peptide and compatible with a pharmaceutical use. In this regard, the invention relates to the use of a polypeptide of the invention as described above for the preparation of pharmacologically active nonpeptide or nonexclusively peptide molecules by determining the structural components of this peptide which are important for its activity and reproducing these components by nonpeptide or nonexclusively peptide structures. The subject of the invention is also pharmaceutical compositions comprising one or more molecules thus prepared.

The subject of the invention is also any pharmaceutical composition comprising, as active ingredient, at least one ligand obtained according to either of the methods described above, and/or at least one antibody or antibody fragment, and/or an antisense oligonucleotide, and/or a compound which are nonexclusively peptide as described above.

The compositions according to the invention may be used for modulating the interaction of the oncogenic mutated forms of p53 with the polypeptides MBP1 or Fibulin 2 and as a result may be used for modulating the proliferation of certain cell types. More particularly, these pharmaceutical compositions are intended for the treatment of diseases involving a cell cycle dysfunction and in particular for the treatment of cancers. They are in particular cancers associated with the presence of oncogenic mutants of p53.

Other advantages of the present invention will emerge on reading the examples which follow and which should be considered as illustrative and nonlimiting.

### LEGEND TO THE FIGURES

FIG. 1: Functional domains of the wild-type p53 protein. TA: transcription activating domain; DNB: DNA binding domain; NLS: nuclear localization signal; OL: oligomerization domain; REG: regulatory domain.

FIG. 2: Interaction between the C-mbp1 protein and the p53 and H175 proteins in mammalian cells.

FIG. 3: Interaction between the C-fibulin2 protein and the p53 and H175 proteins in mammalian cells.

FIG. 4: Comparison of the protein sequences encoded by the mMBP1 (murine) and hMBP1 (human) cDNAs.

FIG. 5: Comparative effects of the C-mbp1 and murine proteins on the cellular growth of tumour cells.

FIG. 6: Expression of the mRNA encoding the MBP1 protein in mice.

FIG. 7: Expression of the mRNA encoding the MBP1 protein in various human tissues.

FIG. 8: Expression of the messenger RNA encoding the human MBP1 protein in colon tumours.

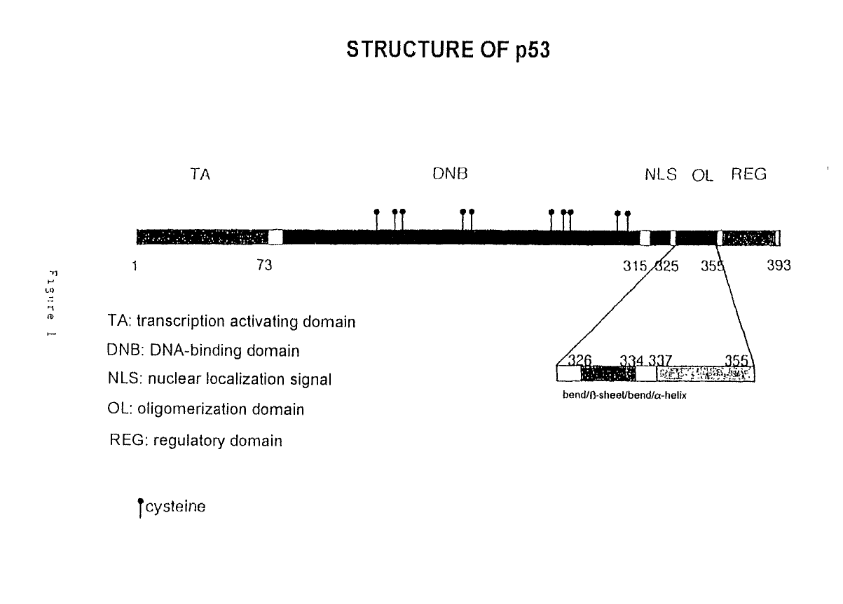

### EXAMPLE 1

### Construction of the various nucleotide fragments necessary for the screening

1-a—Construction of the cDNA Encoding the Human Wild-Type p53

The human p53 gene was cloned by polymerase chain reaction (PCR) on the DNA from a human placenta bank (Clontech) using the 5′-1 and 3′-393 oligonucleotides.

| | |
|---|---|
| 5′-1 oligonucleotide: | |
| AGATCTGTATGGAGGAGCCGCAG | (SEQ ID No. 1) |
| 3′-393 oligonucleotide: | |
| AGATCTCATCAGTCTGAGTCAGGCCCTTC | (SEQ ID No. 2) |

This product was then cloned directly after PCR into the vector pCRII (Invitrogene).

1-b—Construction of the cDNAs Encoding the Various Mutated Forms of Human p53

1-b (i) Construction of the cDNA encoding the H175 mutant of human p53

The cDNA carrying a point mutation on amino acid 175 of the human p53 protein (Arginine→Histidine) was obtained by site-directed mutagenesis on the p53 DNA (described in Example 1-a) by means of the Amersham kit, using the H175 oligonucleotide having the sequence:

| | |
|---|---|
| 3′ H175 oligonucleotide: | |
| GGGGCAGTGCCTCAC | (SEQ ID No. 3) |

This fragment was designated H175.

1-b (ii) Construction of the cDNA encoding the W248 mutant of human p53

The cDNA carrying a point mutation on amino acid 248 of the human p53 protein (Arginine→Tryptophan) was obtained by site-directed mutagenesis on the p53 DNA (described in Example 1-a) by means of the Amersham kit, using the W248 oligonucleotide having the sequence:

| | |
|---|---|
| 3′ W248 oligonucleotide: | |
| GGGCCTCCAGTTCAT | (SEQ ID No. 4) |

This fragment was designated W248.

1-b (iii) Construction of the cDNA encoding the H273 mutant of human p53

The cDNA carrying a point mutation on amino acid 273 of the human p53 protein (Aspartate→Histidine) was obtained by site-directed mutagenesis on the p53 DNA (described in Example 1-a) by means of the Amersham kit, using the H273 oligonucleotide having the sequence:

| | |
|---|---|
| 3′ H273 oligonucleotide: | |
| ACAAACATGCACCTC | (SEQ ID No. 5) |

This fragment was designated H273.

1-b (iv) Construction of the cDNA encoding the G281 mutant of human p53

The cDNA carrying a point mutation on amino acid 281 of the human p53 protein (Asparagine→Glycine) was obtained by site-directed mutagenesis on the p53 DNA (described in Example 1-a) by means of the Amersham kit, using the G281 oligonucleotide having the sequence:

| | |
|---|---|
| 3′ G281 oligonucleotide: | |
| GCGCCGGCCTCTCCC | (SEQ ID No. 6) |

This fragment was designated G281.

1-c—Construction of the cDNAs Encoding Fragments 73-393 of the Wild-Type Human p53 and of the H175 Mutant

1-c (i) Construction of the CDNA encoding fragment 73-393 of the wild-type human p53

This example describe the construction of a cDNA encoding amino acids 73 to 393 of the wild-type human p53 protein (73-393 wt).

This cDNA was obtained by polymerase chain reaction (PCR) on the p53 DNA (described in Example 1-a) with the 3′-393 oligonucleotide (SEQ ID No. 2) and the 5′-73 oligonucleotide below:

| | |
|---|---|
| 5′-73: | |
| AGATCTGTGTGGCCCCTGCACCA | (SEQ ID No. 7) |

1-c (ii) Construction of the cDNA encoding fragment 73-393 of the H175 mutant

This example describes the construction of a cDNA encoding amino acids 73 to 393 of the H175 mutant of the human p53 protein (73-393H175).

This cDNA was obtained by polymerase chain reaction (PCR) on the DNA of the mutant (described in Example 1-b) with the 3′-393 (SEQ ID No. 2) and 5′-73 (SEQ ID No. 7) oligonucleotides.

### EXAMPLE 2

### Construction of the Vectors for Expressing in Yeast Fragments 73-393 wt and 73-393H175 Fused with the DNA Binding Domain of the Gal4 Protein and the Different Forms of the Complete Human p53 (Wild-Type and Mutated) Fused with the Transcription Activating Domain of the Gal4 Protein

This examples describes the construction of vectors allowing the expression, in yeast, of fragments 73-393 wt and 73-393H175 in the form of a fusion with the DNA-binding domain of the Gal4 protein (DB) of the yeast S. cerevisiae for their use in the double-hybrid system and for the screening of CDNA libraries fused with the transcription activating domain (transactivator) of the same Gal4 protein (TA).

2-a—Construction of Yeast Vectors Expressing Fragments 73-393 wt and 73-393H175 Fused with the DNA-Binding Domain of the Gal4 Protein

Fragments 73-393 wt and 73-393H175 were cloned into the vector pPC97 (Chevray et al, Proc. Natl. Acad. Sci. USA 89 (1992) 5789) using the site recognized by the restriction enzyme BglII.

The products of these constructs have the following names:

73-393 wt in pPC97→(plasmid pMA1)→DB-wt

73-393H175 in pPC97→(plasmid pEC16)→DB-H175

2-b—Construction of the Yeast Vectors Expressing the Various Forms of Complete Human p53 (Wild-Type and Mutated) Fused with the Transcription Activating Domain of the Gal4 Protein

The various forms of the complete human p53 (wild-type and mutated) were cloned into the vector pPC86 (Chevray et al, Proc. Natl. Acad. Sci. USA 89 (1992) 5789) using the site recognized by the restriction enzyme BglII.

The products of these constructs have the following names:

p53 in pPC86→(plasmid pEC10)→TA-wt

H175 in pPC86→(plasmid pEC20)→TA-H175

H273 in pPC86→(plasmid pEC87)→TA-H273

G281 in pPC86→(plasmid pEC88)→TA-G281

### EXAMPLE 3

### Cloning by the Double-Hybrid System of the Partners of the H175 Protein, and Characterization of This Interaction in Terms of Specificity in Yeast

This example describes the preparation of partners of H175 protein by the double-hybrid system using the mouse embryo cDNA library pPC67 (Chevray et al, Proc. Natl. Acad. Sci. USA 89 (1992) 5789), and the characterization, with the aid of the same double-hybrid system, of these partners in terms of specificity of interaction with the various forms of the human p53 protein (wild-type and mutated).

3-a—Isolation of the Partners of the H175 Protein

3-a (i)—Genotype of the YCM17 strain

The YCM17 strain used for the isolation of the partners and for the characterization of their interaction with the various forms of the human p53 protein by the double-hybrid system is a strain of yeast of the genus Saccharomyces cerevisiae which has the following genotype:

MATa, Δgal4, Δgal80, lys2, his3, trp1, leu2, ade2, ura3, can1, met16::URA3 pGAL1-10 LacZ.

This yeast strain makes it possible detect a positive response in the double-hybrid system by the appearance of the Ura+ phenotype and/or of the Ura+/LacZ+ double phenotype.

3-a (ii)—Genotype of TG1 strain

The TG1 strain used for the purification of the plasmid DNAs is the bacterial strain of the genus E. coli which has the following genotype:

supE, hsdD5, thi, D(lac-proAB),

F′ [traD36proA⁺B⁻lacI^{q}lacZDM15]

3-a (iii)—Construction of the YMA1 strain

The YCM17 strain was transformed by the method of Gietz et al. (Yeast 11 (1995) 355) with 1 μg of plasmid pMA1 thus allowing the production of YMA1 strain which expresses the DB-H175 protein.

3-a (iv)—Isolation of the partners of the H175 protein

The YMA1 strain was transformed by the same method as that used in Example C1.3 using 100 μg of DNA of the pPC67 library, allowing the production of 3.5×10⁷ transformants among which 404 exhibit the Ura+ phenotype and 14 the Ura+/LacZ+ double phenotype.

The plasmid DNA contained in the 14 clones exhibiting the Ura+/LacZ+ double phenotype was isolated by the method of Ward (Nucl. Acids Res. 18 (1990) 5319) before being used to transform the TG1 strain. The corresponding plasmids derived from the library were then purified and grouped into two subgroups of different plasmids each containing a cDNA encoding two different proteins:

a cDNA encoding the C-terminal part (SEQ ID No. 8) of a new gene,

a cDNA encoding the C-terminal part of murine fibulin 2 (amino acids 863 to 1195 (SEQ ID No. 32)) (Pan et al., J. Cell. Biol. 123 (1993) 1269).

The proteins encoded by these two cDNAs are respectively called C-mbp1 (mbp=′p53 Mutant Binding Protein′) and C-fibulin2, the fusion proteins with the transcription activating domain of Gal4 are named TA-C-mbp1 and TA-C-fibulin2 and the corresponding plasmids are named TA-C-MBP1 and TA-C-FIB2

3-b—Characterization of the Interaction Between the C-mbp1 and C-fibulin2 Proteins and the H175 Proteins in Yeast

3-b (i) Characterization of the specificity of the interaction between the DB-H175 protein and the proteins TA-C-mbp1 and TA-C-fibulin2

With the aim of testing the specificity of the interactions described in Example 3-a (iv), the plasmids pPC86, TA-C-MBP1 and TA-C-FIB2 were reintroduced into the YCM17 strain by cotransformation with various plasmids: the plasmid pPC97 encoding the DB protein, the plasmid pMA1 encoding the DB-H175 protein, the plasmid pEC10 encoding the DB-wt protein and the plasmid pPC76 encoding a fusion protein between the DNA-binding domain of the Gal4 protein and a fragment of the human Fos protein (amino acids 132 to 211) (Chevray et al, Proc. Natl. Acad. Sci. USA, 89 (1992) 5789) (DB-Fos). After the cotransformation, the various clones obtained were tested for the phenotypes associated with the URA3 and LacZ genes.

The results of this experiment are presented in Table 1.

TABLE 1
Specificity of the interaction between the
DB-H175 protein and the proteins TA-C-mbp1 and TA-C-fibulin2

| | TA | TA-C-mbp1 | TA-C-fibulin2 |
|---|---|---|---|
| DB | Ura−/LacZ− | Ura−/LacZ− | Ura−/LacZ− |
| DB-H175 | Ura−/LacZ− | Ura+/LacZ+ | Ura+/LacZ+ |
| DB-wt | Ura−/LacZ− | Ura−/LacZ− | Ura−/LacZ− |
| DE-Fos | Ura−/LacZ− | Ura−/LacZ− | Ura−/LacZ− |

These results show that the interaction between the DB-H175 protein and the proteins TA-C-mbp1 and TA-C-fibulin2 is specific and that such an interaction cannot be obtained either with the DB protein alone or with the DB-wt protein or with the DB-Fos control protein.

3-b (ii)—Construction of fusion proteins between the DNA-binding domain of Gal4 and the proteins C-mbp1 and C-fibulin2

The cDNAs encoding C-mbp1 and C-fibulin2 were extracted from the plasmids TA-C-MBP1 and TA-C-FIB2, and then cloned into the vector pPC97 using the sites recognized by the restriction enzymes SalI and NotI.

The fusion proteins with the DNA-binding domain of Gal4 thus obtained are respectively called DB-C-mbp1 and DB-C-fibulin2 and the corresponding plasmids DB-C-MBP1 and DB-C-FIB2.

3-b (iii) Characterization of the specificity of the interaction between DB-C-mbp1 and DB-C-fibulin2 and the proteins TA-H175 and TA-G281

With the aim of checking that the potential interaction between the proteins C-mbp1 and C-fibulin2 and the H175 protein is not an artefact due to the fusion of one or other of the partners with one or the other of the domains of Gal4, and to confirm the specificity of the interaction with the mutated form of the p53 protein, another interaction experiment in yeast was carried out using fusions different from those of Example 3-b (i).

In this experiment, the proteins DB-C-mbp1 and DB-C-fibulin2 were tested against the fusions of the transcription activating domain of Gal4 with the complete forms of p53 protein (wild-type or mutant) described in Example 2-b, using a yeast strain different from the YCM17 strain, the PCY2 strain (Chevray et al., Proc. Natl. Acad. Sci. USA 89 (1992) 5789).

The results of this experiment are presented in Table 2.

TABLE 2
Specificity of the interaction between the proteins DB-C-mbpl and
DB-C-fibulin2 and the proteins TA-H175 and TA-G281

| | TA | TA-WT | TA-H175 | TA-H273 | TA-G281 |
|---|---|---|---|---|---|
| DB | LacZ− | LacZ− | LacZ− | LacZ− | LacZ− |
| DB-C-mbpl | LacZ− | LacZ− | LacZ+ | LacZ− | LacZ+ |
| DB-C- | LacZ− | LacZ− | LacZ+ | LacZ− | LacZ+ |
| fibulin2 | | | | | |

These results make it possible, on the one hand, to confirm the interaction observed during the screening. On the other hand, these results demonstrate the specificity of the interaction between the proteins C-mbp1 and C-fibulin2 and certain mutated forms of p53 proteins. As regards this specificity, it is of interest to note that these proteins do not interact with the H273 mutant. This is because this mutant exhibits a conformation equivalent to that of the wild-type p53 protein because it is recognized by the antibody PAb 1620 which is specific to the wild-type form and not by the antibody PAb 240 which is specific to the mutated form (Medcalf et al, Oncogene 7 (1992) 71).

Thus, all the data obtained in yeasts show clearly that the two proteins C-mbp1 and C-fibulin2 are potential partners specific to the oncogenic mutants of the p53 protein.

### EXAMPLE 4

### Interaction Between the Proteins C-mbp1 and C-fibulin2 and the Various Forms of the p53 Protein in Mammalian Cells

This example describes the construction of plasmids for the expression of various proteins in mammalian cells and the characterization of the interaction between the proteins C-mbp1 and C-fibulin2 and the various forms of the p53 protein in mammalian, cells.

4-a Construction of the Plasmids for Expressing the Various Proteins in Mammalian Cells

4-a (i) Construction of the expression vector pBFA 107

This example describes the construction of a vector allowing the expression in mammalian cells of proteins carrying a tag derived from the protein c-myc (amino acids 410-419) and recognized by the antibody 9E10 (Oncogene Science). This construction was carried out using, as parent vector, the mammalian expression vector pSV2, described in DNA Cloning, A practical approach Vol. 2, D. M. Glover (Ed) IRL Press, Oxford, Washington D.C., 1985.

The cDNA comprising the sequence encoding the c-myc tag as well as a multiple cloning site (MCS) was constructed from the following 4 oligonucleotides:

| | |
|---|---|
| c-myc 5′: | |
| GATCCATGGAGCAGAAGCTGATCTCCGAGGAGGACCTGA | (SEQ ID No. 10) |
| c-myc 3′: | |
| GATCTCAGGTCCTCCTCGGAGATCAGCTTCTGCTCCATG | (SEQ ID No. 11) |
| MCS 5′: | |
| GATCTCGGTCGACCTGCATGCAATTCCCGGGTGCGGCCGCGAGCT | (SEQ ID No. 12) |
| MCS 3′: | |
| CGCGGCCGCACCCGGGAATTGCATGCAGGTCGACCGA | (SEQ ID No. 13) |

These four oligonucleotides exhibit complementarities in pairs (5′ c-myc/3′ c-myc, 5′ MCS/3′ MCS) and overlapping complementarities (3′ c-myc/5′ MCS) allowing the production of the desired nucleotide sequence by simple hybridization and ligation. These oligonucleotides were phosphorylated with the aid of T4 kinase and then hybridized together and inserted into the expression vector pSV2 previously digested with the restriction enzymes Bgl II and Sac I. The resulting vector is the vector pBFA 107.

4-a (ii) Construction of the plasmids for expressing the tagged proteins C-mbp1 and C-fibulin2

The cDNAs encoding the C-mbp1 and C-fibulin2 proteins were extracted from the plasmids TA-C-MBP1 and TA-C-FIB2 and cloned into the mammalian expression vector pBFA 107 using the sites recognized by the restriction enzymes Sal I and Not I. The plasmids pBFA107-C-MBP1 and pBFA107-C-FIB2 are thus obtained.

4-a (iii) Construction of the plasmids for expressing the various forms of the p53 protein

The cDNAs encoding the various forms of the p53 protein (wt, H175, H273 and G281) were inserted into the expression vectors pSV2 and pcDNA3 (Invitrogen) using the site recognized by the restriction enzyme Bgl II.

4-b—Interaction Between the Proteins C-mbp1 and C-fibulin2 and the Various Forms of the p53 Protein in Mammalian Cells

This example describes the detection in mammalian cells of the interaction between the proteins C-mbp1 and C-fibulin2 and the various forms of the p53 protein. These experiments were carried out by transient transfection and coimmunoprecipitation in H1299 cells (tumour cells of the ‘Non Small Cell Lung Cancer’ type) deficient in the two alleles of the p53 protein (Mitsudomi et al., Oncogene 1 (1992) 171).

The cells (10⁶) were inoculated on Petri dishes, 10 cm in diameter, containing 8 ml of DMEM medium (Gibco BRL) supplemented with 10% heat-inactivated fetal calf serum, and cultured overnight in a CO₂ (5%) incubator at 37° C. The various constructs are then transfected using lipofectAMINE (Gibco BRL) as the transfection agent in the following manner: 6 μg of total plasmid (3 μg of each plasmid encoding each of the two partners) are incubated with 20 μl of lipofectAMINE (Gibco BRL) for 30 min with 3 ml of Opti-MEM medium (Gibco BRL) (transfection mixture). During this period, the cells are rinsed twice with PBS and then incubated for 4 h at 37° C. with the transfection mixture, after which the latter is aspirated and replaced with 8 ml of DMEM medium supplemented with 10% heat-inactivated fetal calf serum and the cells allowed to resume growth at 37° C.

Twenty four hours after the transfection, the cells are washed once with PBS and then scraped, washed again twice with PBS and resuspended in 200 μl of lysis buffer (HNTG: Hepes 50 mM pH 7.5, NaCl 150 mM, Triton x-100 1%, glycerol 10%) supplemented with protease inhibitors (Aprotinin 2 μg/ml, pepstatin 1 μg/ml, leupeptin 1 μg/ml, E64 2 μg/ml and Pefabloc 1 mM), incubated for 30 min at 4° C. and centrifuged for 15 min at 15,000 rpm and 4° C. The cell extract thus obtained is subjected to “pre-clearing” step by incubating for 1 h at 4° C. with 16 μl of pre-immune rabbit serum, and then for 30 min at 4° C. with 200 μl of immunoprecipitin (Gibco BRL) prepared according to the supplier's recommendations. Subsequently, the cell extract thus cleaned is separated into 3 equal batches each of which is incubated overnight at 4° C. with a different antibody; 3 μg of antibody 9E10 (anti myc), 1 μg of antibody DO1 (anti p53) (Oncogene Science) and 1μ of antibody PAb416 (anti SV40 T-Ag, which is used as control antibody) (oncogene Science). This mixture [cell extract/antibody] is then supplemented with 30 μl of immunoprecipitin and incubated for 30 min at 4° C. before being centrifuged for 30 sec at 15,000 rpm. The pellet containing the immunoprecipitin is then washed twice with 1 ml of HNTG buffer supplemented with protease inhibitors, and then resuspended in 30 μl of buffer for loading onto acrylamide gel (Laemmli, Nature 227 (1970) 680) and incubated for 5 min at 95° C. After centrifugation for 15 sec at 15,000 rpm, the supernatants were loaded onto polyacrylamide gel in denaturing medium (Novex) and the proteins separated using the XCell II migration system (Novex) according to the supplier's recommendations, and then transferred onto PVDF membrane (NEN Life Science Products) with the aid of the same XCell II system.

The antibodies 9E10 and DO1 used for revealing the transferred proteins are coupled to biotin LCnHS (Pierce) according to the supplier's recommendations.

The transfer membranes are first of all incubated for 1 h at 4° C. in 10 ml of TTBSN buffer (Tris-HCl 20 mM, pH 7.5, NaCl 150 mM, NaN₃ 0.02%, Tween 20 0.1%) supplemented with 3% bovine serum albumin (BSA) (TTBSN-BSA), and then for 2 h at room temperature with 10 ml of a solution of TTBSN-BSA containing the biotinylated antibody 9E10 (1 μg/ml). After 6 washes with 10 ml of TTBSN buffer, the membranes are then incubated for 1 h at room temperature with 10 ml of a solution of TTBSN-BSA containing ExtrAvidin-Peroxidase (Sigma Immuno Chemicals) diluted to 1/5000, washed again 6 times with TTBSN and treated with the ECL reagent (Amersham) for revealing the proteins by chemiluminescence. The same membranes are then treated with the biotinylated antibody DO1 after having been previously dehybridized (Ellis et al, Nature 343 (1990) 377) and following the same protocol as for the antibody 9E10.

4-b (i)—Interaction between the C-mbp1 protein and the various forms of the p53 protein in mammalian cells

In this example, the H1299 cells were transfected with the following combinations of plasmids before immunoprecipitation and Western blotting:

pBFA107/pBFA107-C-MBP1 (3 μg)+pSV2/pSV2-p53 ( wild-type or mutant) (3 μg)

Furthermore, the following combination serving as control was made:

pBFA107-Sam68 (3 μg)+pSV2-H175 (3 μg)

This control serves to examine whether H175 can or cannot interact either with the myc tag or with a fusion between the myc tag and any protein, the protein Sam68 described by Lock et al. (Cell 84 (1996) 23), not being thought to interact with the various forms of the p53 protein.

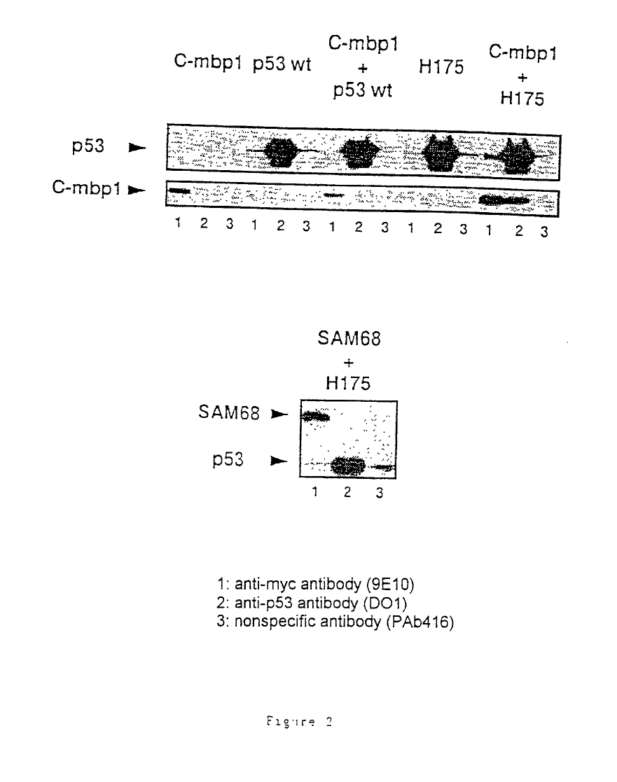

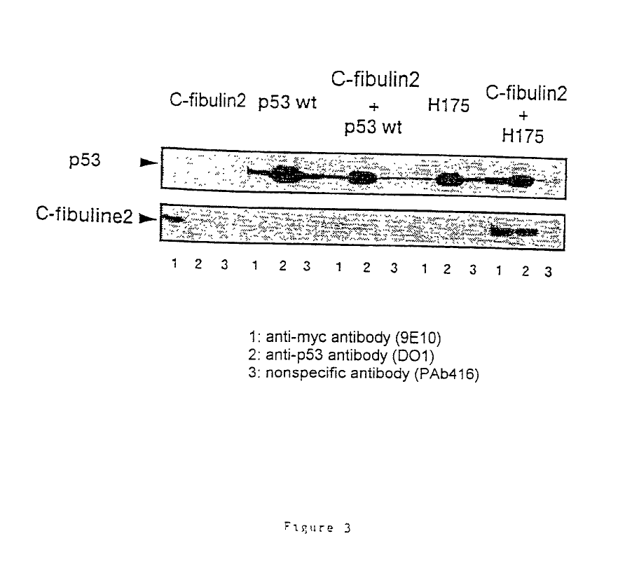

The results of this experiment, which are presented in FIG. 2, show that:

the C-mbp1 protein can interact with the H175 protein in mammalian cells

this interaction is indeed specific to the C-mbp1 protein because the H175 protein does not interact with the myc-Sam68 control

4-b (ii)—Interaction between the C-fibulin2 protein and the various forms of the p53 protein in mammalian cells

In this example, the H1299 cells were transfected with the following combinations of plasmids before immunoprecipitation and Western blotting:

pBFA107/pBFA107-C-FIB2 (3 μg)+pSV2/pSV2-p53 (wild-type or mutant) (3 μg)

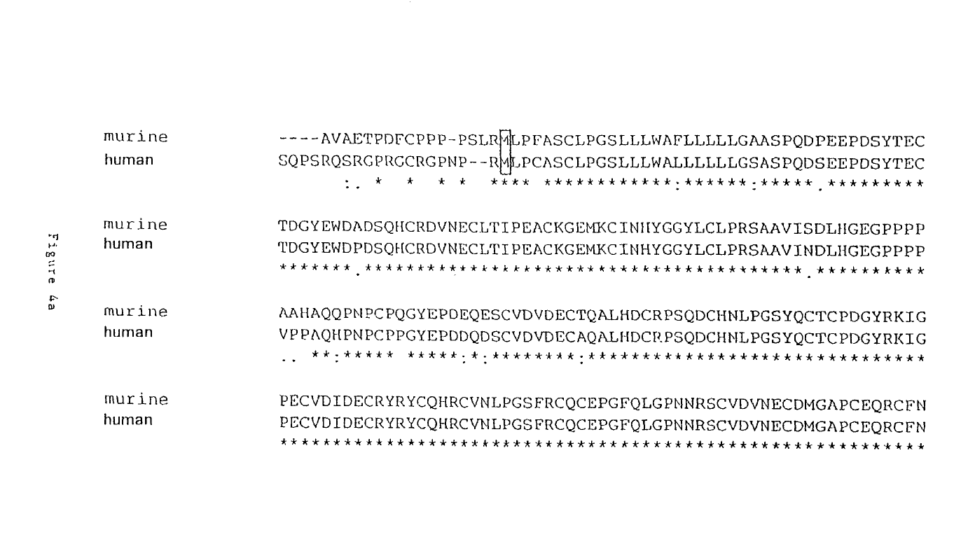

The results of this experiment, which are presented in FIG. 3, show that the C-fibulin2 protein can interact specifically with the H175 protein in mammalian cells.

The general conclusion to these experiments is that the proteins C-mbp1 and C-fibulin2 are capable of interacting specifically in mammalian cells with the H175 proteins. These results, like those obtained in yeast (Example 3), are in agreement with:

1/ the classification of the mutants of the p53 protein:

H175 and G281: dominant-oncogenic

H273: weak mutant

2/ the classification of the various forms of the p53 protein in terms of conformation and recognition by conformational antibodies:

H175 and G281: mutant conformation, PAb 1620−/PAb 240+

p53 and H273: wild-type conformation, PAb 1620+/Pab 240−

All these data show that the proteins C-mbp1 and C-fibulin2 interact with the forms of the p53 protein having a mutated conformation, and that they are capable of having an effect on specific functions of the mutated forms of the p53 protein.

Furthermore, from the literature, it is known that a fraction of the p53 protein may exhibit a mutant conformation in mammalian cells, in particular:

1/ the p53 protein, capable of binding to DNA (Hupp et al, Nucl. Acids Res. 21 (1993) 3167), may adopt a mutant conformation when it binds to DNA (Halazonetis et al, EMBO J. 12 (1993) 1021)

2/ the p53 protein may adopt two different conformations during the cell cycle; the so-called “suppressor” conformation (wild-type conformation, PAb 1620+/PAb 240−) and the so-called ‘promoter’ conformation (mutant conformation, PAb 1620−/PAb 240+) (Milner & Watson, Oncogene 2 (1990) 1683).

It can therefore be assumed that the proteins C-mbp1 and C-fibulin 2 are also capable of having an effect on specific functions of the wild-type form of the p53 protein.

### EXAMPLE 5

### Effect of the C-mbp1 Protein on the Oncogenic Cooperation Between the H175 Protein and the Ras-Val12 Protein

This example describes the effects of the C-mbp1 protein on a property of the oncogenic mutant H175, its capacity to cooperate with the mutated form of the Ras proto oncogene (Ras-Val12) in the oncogenic transformation of rat embryonic fibroblasts.

The rat embryonic fibroblasts (REF) were prepared from OFA rats (IFA-CREDO) according to the method described by C. Finlay (Methods in Enzymology 255 (1995) 389). After thawing, the cells (1.5×10⁶) are inoculated on Petri dishes, 10 cm in diameter, containing 8 ml of DMEM medium (Gibco BRL) supplemented with 10% fetal calf serum and cultured overnight in a CO₂ (5%) incubator at 37° C., and are then transfected with the various mixtures of plasmids (21 μg of DNA) using the CellPhect reagent (Pharmacia) according to the supplier's recommendations. 24 h after the end of the transfection, the cells contained in each of the dishes are scraped and then reinoculated on three 10-cm Petri dishes and cultured for 15 days before being stained with crystal violet according to the protocol described by C. Finlay (Methods in Enzymology 255 (1995) 389). The transformation foci are then visualized and counted.

The plasmids used during this series of experiments are the following:

buffer plasmid: pSG5 (Stratagene)

plasmid for expressing the Ras-Val12 protein: pEJ-Ras (Shih & Weinberg, Cell 29 (1982) 161)

plasmid for expressing the complete c-myc protein: pSVc-myc1 (Land et al, Nature 304 (1983) 596)

plasmid for expressing the H175 protein: pSV2-H175 (Example 4-a (iii))

plasmid for expressing the C-mbp1 protein: pBFA107-C-MBP1 (Example 4-a (ii))

Each transfection spot contains a mixture of three plasmids in an amount of 7 μg of each plasmid. The results of two independent experiments are presented in Table 3.

TABLE 3
Effect of the C-mbp1 protein on the oncogenic cooperation
between the H175 protein and the Ras-Val12 protein

| | Experiment 1 | Experiment 2 |
|---|---|---|
| Control | 0 | 0 |
| Ras-Val12 | 0 | 0 |
| c-myc | 0 | NT |
| H175 | 0 | NT |
| C-mbp1 | 0 | NT |
| Ras-Val12 + c-myc | 111 | 16* |
| Ras-Val12 + c-myc + C-mbp1 | 113 | 12* |
| Ras-Val12 + H175 | 0 | 16 |
| Ras-Val12 + C-mbp1 | 0 | 3 |
| Ras-Val12 + H175 + C-mbp1 | 13 | 30 |

(NT: Not tested *:experiment carried out with 3 μg of plasmid pSVc-myc1)

The results of these experiments show that:

C-mbp1 can cooperate with the activated form of Ras for the transformation of REFs

there is synergy between the H175 and C-mbp1 proteins in the oncogenic cooperation with Ras which is specific for this association because C-mbp1 has no effect on the Ras/c-myc oncogenic cooperation.

### EXAMPLE 6

### Effect of the Proteins C-mbp1 and C-fibulin2 and the Relationship With the Effects of the Various Forms of the p53 Protein on the Cellular Growth of Tumour Cells

This example describes the effects of the proteins C-mbp1 and C-fibulin2 on the cellular growth of tumour cells and their relationship with the effects of the various forms of the p53 protein on the same cellular growth.

These effects of the proteins C-mbp1 and C-fibulin2 on cellular growth were tested on the H1299 cell line in an experiment for forming colonies resistant to neomycin following transfection with plasmids expressing these proteins.

These transfection experiments were carried out according to the protocol described in Example 4-b using 10⁵ cells per spot and 1.5 μg of total DNA.

The plasmids used during this series of experiments are the following:

plasmids for expressing the p53 and H175 proteins: pSV2-p53 and pSV2-H175.

plasmid for expressing the C-mbp1 protein: pBFA107-C-MBP1 (Example 4-a(ii))

plasmid for expressing the C-fibulin2 protein: pBFA107-C-FIB2 (Example 4-a (ii))

plasmid conferring resistance to neomycin: pSV2-Neo for a total quantity of 0.4 μg

Protocol for forming colonies resistant to neomycin: 48 h after transfection, the cells are scraped and transferred onto two Petri dishes, 10 cm in diameter, and placed in culture again with 10 ml of DMEM medium supplemented with 10% heat-inactivated fetal calf serum and containing 400 μg/ml of geneticin (G418). After selecting for 15 days in the presence of G418, the number of Neo^{R} colonies is determined by counting after staining with fuchsin.

The results of these experiments are presented in Tables 4 and 5.

TABLE 4
Effect of the C-mbp1 protein on the cellular growth of tumour cells

| | Number of colonies resistant to Neomycin | | | |
|---|---|---|---|---|
| Protein | Experiment | Experiment | Experiment | |
| expressed | 1 | 2 | 3 | Mean |
| Vector | 36 (1.00) | 52 (1.00) | 73 (1.00) | 1.00 |
| C-mbp1 100 ng | 45 (1.25) | 49 (1.06) | 69 (0.95) | 1.09 |
| C-mbp1 500 ng | 51 (1.42) | 71 (1.37) | 110 (1.51) | 1.43 |
| C-mbp1 1000 ng | 70 (1.94) | 83 (1.60) | 160 (2.19) | 1.90 |
| Wild-type p53 100 ng | 7 (0.19) | 12 (0.23) | 10 (0.14) | 0.19 |
| C-mbp1 100 ng | 6 (0.17) | 14 (0.27) | 8 (0.11) | 0.18 |
| C-mbp1 500 ng | 19 (0.53) | 28 (0.54) | 23 (0.32) | 0.46 |
| C-mbp1 1000 ng | 32 (0.89) | 50 (0.96) | 51 (0.70) | 0.85 |
| Wild-type p53 200 ng | 2 (0.06) | 5 (0.10) | 8 (0.11) | 0.08 |
| C-mbp1 100 ng | 2 (0.06) | 4 (0.08) | 6 (0.08) | 0.08 |
| C-mbp1 500 ng | 5 (0.14) | 8 (0.15) | 16 (0.22) | 0.17 |
| C-mbp1 1000 ng | 9 (0.25) | 20 (0.38) | 28 (0.38) | 0.35 |
| H175 100 ng | 41 (1.14) | 47 (0.90) | 61 (0.84) | 0.96 |
| C-mbp1 100 ng | 33 (0.92) | 65 (1.25) | 70 (0.96) | 1.04 |
| C-mbp1 500 ng | 67 (1.86) | 101 (1.94) | 123 (1.68) | 1.83 |
| C-mbp1 1000 ng | 162 (4.50) | 128 (2.46) | 316 (4.33) | 3.76 |
| H175 200 ng | 39 (1.08) | 60 (1.15) | 66 (1.10) | 1.11 |
| C-mbp1 100 ng | 43 (1.19) | 54 (1.04) | 75 (1.03) | 1.10 |
| C-mbp1 500 ng | 59 (1.64) | 129 (2.48) | 163 (2.23) | 2.12 |
| C-mbp1 1000 ng | 131 (3.64) | 282 (5.42) | 299 (4.10) | 4.39 |

TABLE 5
Effect of the C-fibulin2 protein on the cellular growth of tumour cells

| | Number of colonies resistant to Neomycin | | | |
|---|---|---|---|---|
| Protein | Experiment | Experiment | Experiment | |
| expressed | 1 | 2 | 3 | Mean |
| Vector | 36 (1.00) | 52 (1.00) | 73 (1.00) | 1.00 |
| C-fibulin2 100 ng | 35 (0.97) | 56 (1.08) | 80 (1.10) | 1.05 |
| C-fibulin2 500 ng | 48 (1.33) | 68 (1.31) | 102 (1.40) | 1.35 |
| C-fibulin2 1000 ng | 60 (1.67) | 87 (1.67) | 194 (2.66) | 2.00 |
| Wild-type p53 100 ng | 7 (0.19) | 12 (0.23) | 10 (0.14) | 0.19 |
| C-fibulin2 100 ng | 10 (0.28) | 11 (0.21) | 13 (0.18) | 0.22 |
| C-fibulin2 500 ng | 15 (0.42) | 30 (0.58) | 26 (0.36) | 0.45 |
| C-fibulin2 1000 ng | 35 (0.97) | 44 (0.85) | 45 (0.62) | 0.81 |
| Wild-type p53 200 ng | 2 (0.06) | 5 (0.10) | 8 (0.11) | 0.09 |
| C-fibulin2 100 ng | 3 (0.08) | 6 (0.12) | 6 (0.08) | 0.09 |
| C-fibulin2 500 ng | 4 (0.11) | 10 (0.19) | 16 (0.22) | 0.16 |
| C-fibulin2 1000 ng | 10 (0.28) | 18 (0.35) | 28 (0.38) | 0.34 |
| H175 100 ng | 41 (1.14) | 47 (0.90) | 61 (0.84) | 0.96 |
| C-fibulin2 100 ng | 47 (1.31) | 54 (1.04) | 84 (1.15) | 1.17 |
| C-fibulin2 500 ng | 84 (2.33) | 95 (1.83) | 156 (2.14) | 2.10 |
| C-fibulin2 1000 ng | 143 (3.97) | 138 (2.65) | 270 (3.70) | 3.44 |
| H175 200 ng | 39 (1.08) | 60 (1.15) | 66 (1.10) | 1.11 |
| C-fibulin2 100 ng | 51 (1.42) | 63 (1.21) | 80 (1.10) | 1.24 |
| C-fibulin2 500 ng | 74 (2.06) | 146 (2.81) | 142 (1.95) | 2.27 |
| C-fibulin2 1000 ng | 158 (4.39) | 230 (4.42) | 284 (3.89) | 4.23 |

The results of these experiments show that:

the proteins C-mbp1 and C-fibulin2 have a positive effect on cellular growth

the proteins C-mbp1 and C-fibulin2 are capable of blocking the antiproliferative effect of the p53 protein, independently of their proliferative effect

the proliferative effect of the proteins C-mbp1 and C-fibulin2 is greatly increased in the presence of the H175 protein.

### EXAMPLE 7

### Cloning of the cDNAs Encoding the Complete Form of the Murine and Human MBP1 Proteins

This example describes the cloning of the cDNAs encoding the complete murine MbP1 protein and the use of these data for the cloning of a human homolog of MBP1.

7 a—Cloning of the cDNA Encoding the Complete Form of the Murine mbp1 Protein

The cDNA encoding the C-terminal part of the murine mbp1 protein was cloned by polymerase chain reaction (PCR) on the DNA of the murine embryo SuperScript library (8.5 days) (Gibco BRL) using the 3′-mMBP1 oligonucleotide and the SP6 oligonucleotide (Gibco BRL).

| | |
|---|---|
| 3′-mNBP1 oligonucleotide: | |
| CGGTACTGGCAGAGGTAACTGG | (SEQ ID No. 14) |

This amplification made it possible to obtain a single product having a size of about 800 base pairs which was then cloned directly after PCR into the vector pCRII (Invitrogen) and sequenced. The sequence thus obtained (SEQ ID No. 15) shows an overlapping of 368 base pairs with C-MBP1 (SEQ ID No. 8) with a strict sequence identity on this common portion. Furthermore, in 5′ of this common portion, an additional sequence of 445 base pairs exists having an open reading frame and a codon for initiation of translation.

The two fragments represented by these sequences were then assembled by a three-partner ligation using the sites recognized by the restriction enzymes EcoR I, Pst I and Not I and the plasmid pBC−SK+ (STRATAGENE) thus allowing the reconstitution of the complete murine MBP1 cDNA (mMBP1) (SEQ ID No. 16).

7-b Cloning of the cDNA Encoding the Complete Form of the Human mbp1 Protein

The sequence of the murine MBP1 gene was used for a search for homology in Genbank. This search made it possible to show a strong homology with the sequence of a human EST (g1548384). From this sequence, two cDNA fragments were cloned by polymerase chain reaction (PCR) on the DNA of the human testicle SuperScript library (Gibco BRL) using the 3′-hMBP1 and SP6 oligonucleotides (Gibco BRL), on the one hand, and the 5′-hMBP1 and T7 oligonucleotides (Gibco BRL), on the other hand.

| | |
|---|---|
| 3′-hMBP1 oligonucleotide: | |
| CTCCGCTCCGAGGTGATGGTC | (SEQ ID No. 17) |
| 5′-hMBP1 oligonucleotide: | |
| TGTAGCTACTCCAGCTACCTC | (SEQ ID No. 18) |

These amplifications made it possible to obtain two products having sizes of about 1100 and 700 base pairs which were then cloned directly after PCR into the vector pCRII (Invitrogene) and sequenced. The sequences thus obtained (SEQ ID No. 19 and SEQ ID No. 20) show an overlapping of 325 pairs with a strict sequence identity on this common portion.

The two fragments represented by these sequences were then assembled by a three-partner ligation using the sites recognized by the restriction enzymes EcoR I, Nco I and Not I and the plasmid pBC−SK+ (STRATAGENE) thus allowing the reconstitution of the complete human MBP1 cDNA (hMBP1) (SEQ ID No. 21) having an open reading frame and a codon for initiation of translation.

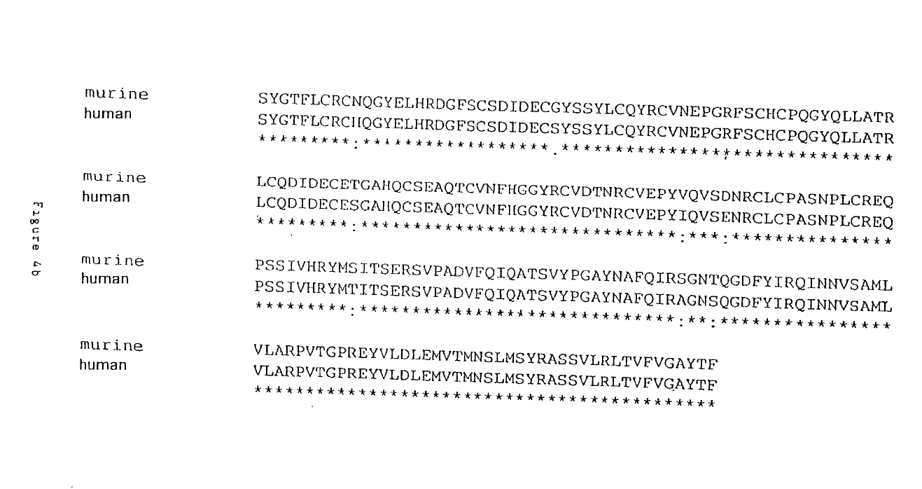

The comparison of the protein sequences corresponding to the cDNAs previously obtained (Examples 7-a and 7-b) (FIG. 4) show a strict identity of 95% in the presumed open reading frame (after the presumed site for initiation of translation (ATG)). On the other hand, an absence of identity and a very poor homology are observed between the regions situated upstream of this putative site for initiation of translation (ATG). These data therefore make it possible to confirm this position as initiating translation and hence that these two cDNAs indeed encode the complete forms of the human (SEQ ID No. 22) and murine (SEQ ID No. 16) MBP1 proteins.

7-c Construction of the Plasmids Expressing in Mammalian Cells the Murine and Human Forms of the mbp1 Protein

The cDNAs encoding the murine and human forms of the MBP1 protein contained in the vector pBC SK+ were inserted into the expression vectors pSV2 and pcDNA3 (Invitrogen) using the sites recognized by the restriction enzymes HindIII and NotI.

### EXAMPLE 8

### Comparative Effects of the Murine C-mbp1 and mbp1 Proteins on the Cellular Growth of Tumour Cells

This example describes the comparative effects of the murine C-mbp1 and mbp1 proteins on the cellular growth of tumour cells and their relationship with the effects of the H175 protein on this same cellular growth.

These effects of the murine mbp1 protein on cellular growth were tested on the H1299 cell line in an experiment for forming colonies resistant to neomycin following transfection with plasmids carrying the cDNA encoding these proteins.

These transfection experiments were carried out according to the protocol described in Example D2 using 10⁵ cells per spot and 1.5 μg of total DNA.

The plasmids used during this series of experiments are the following:

plasmid for expressing the H175 protein: pSV2-H175.

plasmid for expressing the C-mbp1 protein:

pBFA107-C-MBP1 (Example 4-a (ii))

plasmid for expressing the murine mbp1 protein:

pSV2-mMBP1 (Example 7-c)

plasmid conferring resistance to neomycin: pSV2-Neo for a total quantity of 0.4 μg

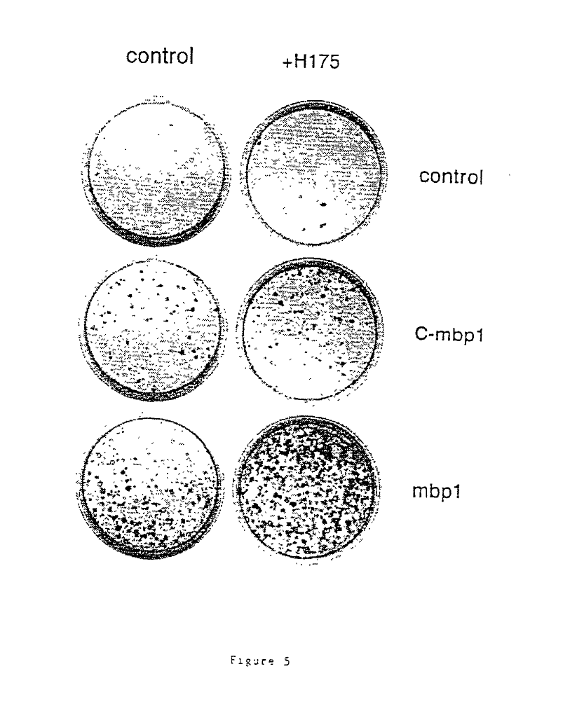

The protocol for forming colonies resistant to neomycin which is used is that described in Example 6. The results of this experiment are presented in Table 6 and FIG. 5.

TABLE 6
Comparative effects of the murine C-mbp1 and
mbp1 proteins on the cellular growth of tumor cells

Number of colonies resistant
to Neomycin

| Protein expressed | Experiment 1 | Experiment 2 |
|---|---|---|
| Vector | 61 (1.00) | 71 (1.00) |
| C-mbp1 100 ng | 67 (1.10) | |
| C-mbp1 500 ng | 96 (1.57) | |
| C-mbp1 1000 ng | 278 (4.56) | 239 (3.37) |
| mbp1 100 ng | 94 (1.54) | |
| mbp1 500 ng | 128 (2.10) | |
| mbp1 1000 ng | 419 (6.87) | 562 (7.92) |
| H175 200 ng | 65 (1.07) | 69 (0.97) |
| C-mbp1 100 ng | 72 (1.18) | |
| C-mbp1 500 ng | 134 (2.20) | |
| C-mbp1 1000 ng | 397 (6.51) | 341 (4.80) |
| mbp1 100 ng | 81 (1.33) | |
| mbp1 500 ng | 206 (3.38) | |
| mbp1 1000 ng | 729 (11.95) | 1215 (17.11) |

The results of this experiment show that the murine mbp1 protein exhibits the same characteristics as the C-mbp1 protein, namely a positive effect on cellular growth which is substantially increased in the presence of the H175 protein.

Furthermore, this effect of mbp1 protein is very substantially increased relative to the truncated C-mbp1 protein.

### EXAMPLE 8a

### Oncogenic Cooperation of the Murine C-mbp1 and mbp1 and Human mbp1 Proteins With the Ras-Val12 Protein

This example describes the comparative effects of the murine C-mbp1 and mbp1 and human mbp1 proteins in an experiment for oncogenic cooperation with Ras-Val12 protein.

This oncogenic cooperation was tested on rat embryonic fibroblasts following transfection with plasmids carrying the cDNAs encoding these proteins and following the protocol described in Example 5.

The results of this experiment are presented in Table 7.

TABLE 7
Oncogenic cooperation of the murine C-mbp1 and
mbp1 and human mbp1 proteins with the Ras-Val12 protein

| | Experiment 1 | Experiment 2 |
|---|---|---|
| Control | 0 | 0 |
| Ras-Val12 | 0 | 0 |
| C-myc | 0 | 0 |
| H175 | 0 | 0 |
| C-mbp1 | 0 | 0 |
| mbp1 | 0 | 0 |
| Ras-Val12 + c-myc | 31 | 42 |
| Ras-Val12 + H175 | 10 | 15 |
| Ras-Val12 + C-mbp1 | 4 | 4 |
| Ras-Val12 + murine mbp1 | 5 | 7 |
| Ras-Val12 + human mbp1 | 6 | 6 |

The results of this experiment show that the murine MBP1 and human MBP1 proteins have the same characteristics as the C-mbp1 protein, namely the capacity to cooperate with the Ras-Val12 protein for the transformation of rat embryonic fibroblasts.

Advantageously, it is also noted that the fibroblasts thus transformed exhibit a quite particular morphological appearance which differs from that obtained with the c-myc oncogene.

### EXAMPLE 9

### Expression of the mbp1 Protein in Mice and in Human Tissues

This example describes the study of the expression of the MBP1 messenger RNA in mice and in various human tissues.

9-a Preparation of the probes

The mMBP1 and hMBP1 probes consist of the corresponding cDNAs.

The GAPDH probe (control) was generated by polymerase chain reaction (PCR) on the DNA of the human testicle SuperScript bank (Gibco BRL) (GAPDH) using the following oligonucleotides:

| | |
|---|---|
| sense-GAPDE oligonucleotide: | |
| CGGAGTCAACGGATTTGGTCGTAT | (SEQ ID No. 24) |
| antisense-GAPDH oligonucleotide: | |
| AGCCTTCTCCATGGTGGTGAAGAC | (SEQ ID No. 25) |

The probes were radiolabelled with ³²P-dCTP using the Rediprime kit (Amersham) and the supplier's recommendations, and the nonincorporated nucleotides were eliminated by chromatography on MicroSpin G-25 columns (Pharmacia Biotech). The Northern blots used during this experiment were obtained from Clontech. The membranes were prehybridized with the ExpressHyb solution (Clontech) for 45 minutes at 65° C. and then incubated for 2 hours with the radiolabelled probes at 65° C., washed three times with 2×SSC buffer, twice with 2×SSC buffer supplemented with 0.1% SDS and finally washed with 0.2×SSC buffer supplemented with 0.1% SDS until the background noise disappeared. The membranes were then subjected to autoradiography and quantification of the signal was carried out with the aid of an instantimager (Packard instruments).

9-b Expression of the MBP1 protein in mice

This Example Describes the Study of the Expression of the MBP1 Messenger RNA in Mice.

The probes used in this experiment are the mMBP1 and GAPDH probes. The membranes used in this experiment contain one of the mouse embryo mRNAs obtained at various stages of development, and the other of the mRNAs representative of various adult mouse tissues.

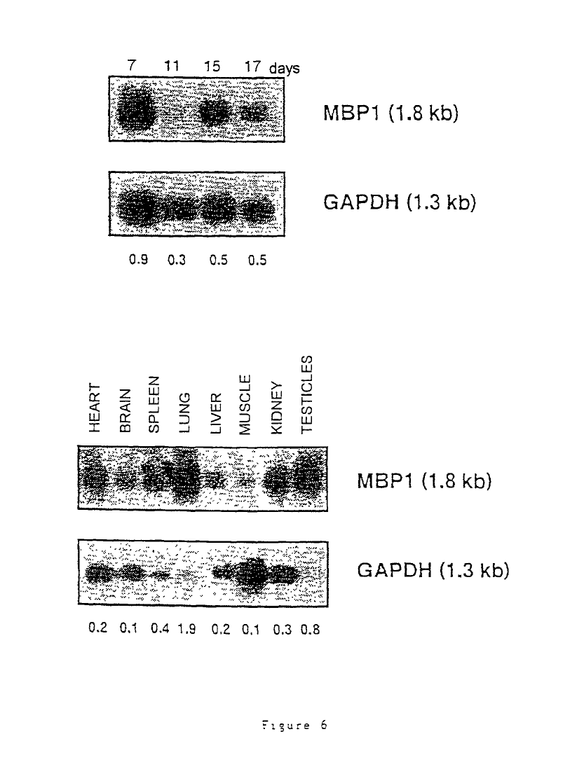

The results of this experiment (FIG. 6) clearly indicate that:

1—a single transcript of 1.8 kb is detected both in the mouse embryo mRNAs and in the adult mouse tissues.

2—this messenger exhibits variations in the levels of expression during development, with a high abundance in the early stages (7 days) and then a substantial reduction (11 days), reaching an apparently constant level.

3—this messenger is moderately expressed in all the adult tissues tested with the exception of a substantial expression in the lungs and the testicles.

A high level of expression of transcript of this kind in a phase of embryo development as well as in tissues exhibiting a high growth rate confirms the involvement of the product of the MBP1 gene in the processes of cellular growth identified in Examples 5, 6 and 7.

9-c Expression of the MBP1 Protein in Human Tissues

This example describes the study of the expression of the MBP1 messenger RNA in various human tissues.

The probes used in this experiment are the hMBP1 and GAPDH probes. The membranes used contain mRNAs representative of various human tissues.

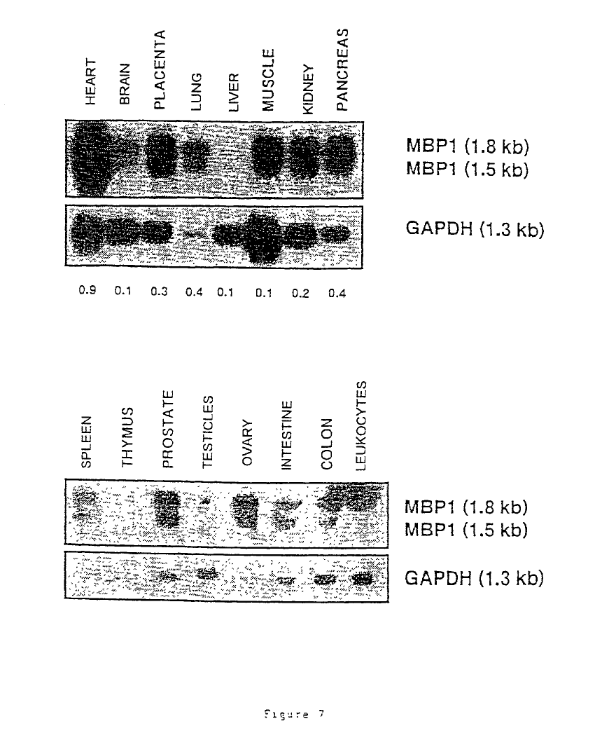

The results of this experiment (FIG. 7) clearly indicate that:

1—two transcripts of 1.5 and 1.8 kb are detected in the human tissues.

2—these messengers are moderately expressed in all the tissues tested and their expression profile is comparable with that of the murine messenger (high expression in the lungs and the testicles).

These results show that:

two different forms of the human MBP1 protein may exist with the possibility of alternative splicing of the messenger.

the mRNA encoding the human mbp1 protein(s), just like their murine homolog, exhibit a high level of expression in tissues at a high growth rate, and that the product(s) of the human MBP1 gene may therefore also be involved in the cell growth processes.

The results presented in the various examples show that the C-mbp1, MBP1 and C-fibulin2 proteins interact specifically with the mutant forms of the p53 protein and that these interactions lead to a synergy between these proteins and the oncogenic mutants of the p53 protein whether for oncogenic cooperation with the activated form of the Ras protein or for the proliferative effect.

Furthermore, the C-mbp1, MBP1 and C-fibulin2 proteins exhibit an intrinsic proliferative activity, and the C-mbp1 protein acts as an immortalizing oncogene by cooperating with the activated form of the Ras protein for cell transformation.

The results presented in the various examples show that the proteins or polypeptides C-mbp1, MBP1 and C-fibulin2 interact specifically with the mutant forms of the p53 protein and that these interactions lead to a synergy between these proteins and the oncogenic mutants of the p53 protein whether for oncogenic cooperation with the activated form of the Ras protein or for the proliferative effect.

Furthermore, the C-mbp1, MBP1 and C-fibulin2 proteins exhibit an intrinsic proliferative activity, and the C-mbp1 and MBP1 proteins act as immortalizing oncogenes by cooperating with the activated form of the Ras protein for cell transformation.

These properties confer on MBP1 a potential role as oncogene. In at least one of the tests (Example 8a), the MBP1 protein exhibits increased oncogenic properties compared with the c-MBP1 polypeptide.

Finally, the strong homology exhibited by the human and murine MBP1 proteins (95% strict identity), and the similarity in tissue expression of their respective messengers, make it possible to conclude that the human MBP1 protein, which may be present in the form of two different variants (2 distinct messengers), possess(es) properties similar to those of its murine homolog.

In conclusion, these results describe the characterization of a new murine protein, MBP1, and of its human homolog(s), which exhibits oncogenic properties and which interacts specifically with the mutated forms of the p53 protein. This interaction which results in an increase in the oncogenic properties of MBP1 could constitute a key element of the oncogenic capacity of these mutants of the p53 protein.

Such properties appear to be also shared by another protein exhibiting homologies with MBP1, fibulin 2. Since this protein is part of a larger family, it is possible to envisage that these properties can be extended to all the members of the fibulin family.

Since these interactions show a strong synergy between the oncogenic powers of the proteins MBP1, fibulin2 and p53 mutants, they constitute a potential point of action in the treatment of cancers linked to mutations in the p53 protein. Furthermore, the MBP1 and fibulin2 proteins which exhibit intrinsic oncogenic properties constitute potential targets for the treatment of cancer in general.

### EXAMPLE 10

### Chromosomal Location of the Human MBP1 Gene

The chromosomal location of the MBP1 gene was obtained according to a four-step protocol (Lichter et al, Science 247 (1990) 64) (Heng et al, Chromosoma 102 (1993) 325) (Kischkel et al, Cytogenet. Cell Genet. 82 (1998) 95):

labelling of the cDNA with biotin by nick translation

hybridization on normal human metaphases (high-resolution technology)

revealing with fluorescein

visualization and interpretation on an epifluorescence microscope

This study of hybridization of the MBP1 probe on human metaphases was carried out by analysing 30 mitoses and showed the presence of a double spot on the long arms (q arm) of the two chromosomes 11 in 11q13. Interestingly, this region of chromosome 11 has been associated with a large number of pathological conditions:

Mac Ardle's disease (Lebo et al, Science 225 (1984) 57)

Usher's syndrome type 1B (Weil et al, Nature 374 (1995) 60)

endocrine neoplasia type I (Teh et al, J. Intern. Med. 238 (1995) 249)

Best's dystrophy (Graff et al, Genomics 24 (1994) 425)

insulin-dependent diabetes (Davies et al, Nature 371 (1994) 130)

spinocerebellar ataxia 5 (Ranum et al, Nature Genet. 8 (1994) 280)

Bardet-Biedl's syndrome (Leppert et al, Nature Genet. 7 (1994) 108)

osteoporosis (Gong et al, Am. J. Hum. Genet. 59 (1996) 146)

Furthermore, this region of chromosome 11 is also the site of amplification events associated with various solid tumours (oesophagus, head and neck, bladder, breast and lung) (Lammie & Peters, Cancer Cells 3 (1991) 413).

The MBP1 gene could therefore not only be associated with a certain number of cancers but also with a large number of pathological conditions exhibiting disorders of the renal, neurodegenerative and bone types, and the like. Among these pathological conditions, there may be mentioned in particular: acute renal deficiencies such as those associated with Mac Ardle's disease, retinis pigmentosa and certain forms of blindness and deafness such as those associated with Usher's syndrome type 1B, hyperthyroidism such as the form associated with endocrine neoplasia type I, pathologies linked to a retinal pigmentation defect such as those encountered in Best's dystrophy, insulin-dependent diabetes, neurodegenerative pathological conditions such as those associated with cerebrospinal ataxia 5, retinal dystrophies, renal disorders such as the forms encountered in Bardet-Biedl's syndrome, and osteoporosis.

### EXAMPLE 11

### Expression of the Messenger RNA Encoding the Human MBP1 Protein in Colon Yumours

This example describes a semi-quantitative analysis of the expression of the messenger RNA encoding the human MBP1 protein, carried out in parallel on 9 colon tumours and 9 healthy tissue samples (colon) obtained from the same patients.

The samples were frozen at −70° C. immediately after resection and the total RNA was prepared by homogenizing 100 mg of tissue using the RNA NOW solution (Ozyme) and the protocol recommended by the supplier. Subsequently, cDNA synthesis was carried out with the aid of the First-Strand cDNA Synthesis kit (Amersham Pharmacia Biotech) using 1.5 μg of total RNA and according to the supplier's recommendations. The MBP1 and β-actin (control) genes were amplified by PCR using a quantity of cDNA for which the level of PCR product can be directly correlated with the concentration of substrate and the following programme of cycles:

| | |
|---|---|
| 1 cycle | 2 min at 95° C. |
| 30 cycles | 30 sec at 94 |
| | 1 min at 45° C. |
| | 1 min at 72° C. |
| 1 cycle | 3 min at 72° C. |

The oligonucleotides used for these amplifications are the following:

| | |
|---|---|
| sense-MBP1 oligonucleotide | |
| GCCCTGATGGTTACCGCAAGA | (SEQ ID No. 27) |
| antisense-MBP1 oligonucleotide | |
| AGCCCCCATGGAAGTTGACAC | (SEQ ID No. 28) |
| sense-β-actin oligonucleotide | |
| GTGGGGCGCCCCAGGCACCA | (SEQ ID No. 29) |
| antisense-β-actin oligonucleotide | |
| CGGTTGGCCTTGGGGTTCAGGGGGG | (SEQ ID No. 26) |

The PCR products thus generated were then analysed by electrophoreses on a 1% agarose gel.

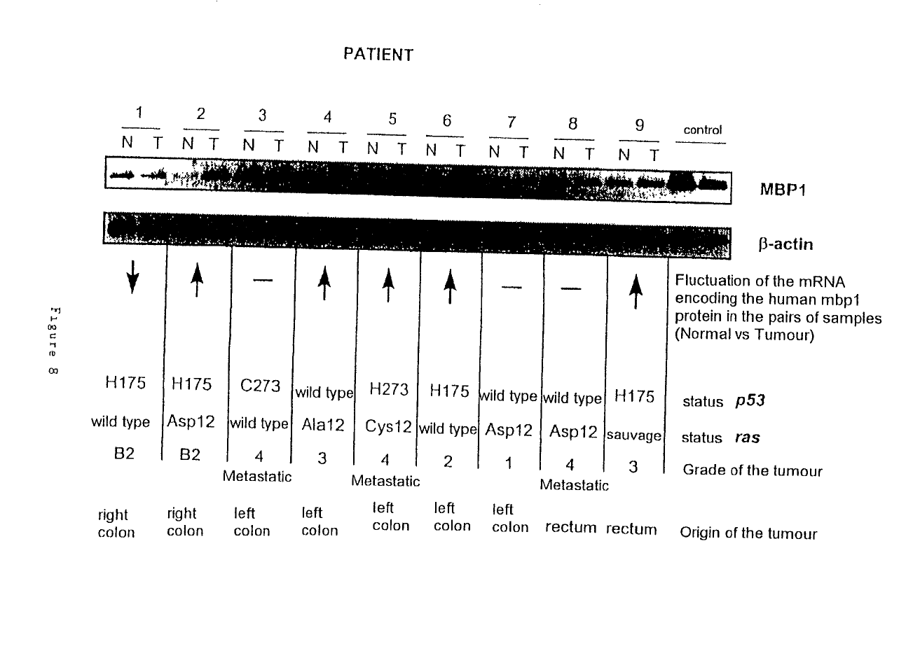

The results presented in FIG. 8 clearly show that the messenger RNA encoding the human MBP1 protein is amplified in five of the tumours studied compared with the healthy tissue obtained from the same patient, this being regardless of the grade of the tumour and independently of their status as regards the Ras and p53 genes.

The results of this example therefore show that amplification of the messenger RNA encoding the human MBP1 protein maybe detected in some types of human tumours and therefore highlight a potential role of the MBP1 protein in the appearance and/or development of these tumours.

1. Polypeptide capable of interacting specifically with the oncogenic forms of p53, of stimulating cell growth and of blocking the antiproliferative effects of the wild-type form of p53.

2. Polypeptide according to claim 1, characterized in that it comprises all or part of a sequence chosen from the polypeptide sequences SEQ ID No. 9 or SEQ ID No. 16 or a derivative thereof.

3. Polypeptide according to claim 1, characterized in that it comprises all or part of a sequence chosen from the polypeptide sequences SEQ ID No. 31 or SEQ ID No. 22 or a derivative thereof.

4. Polypeptide according to claim 1, characterized in that it comprises all or part of the polypeptide sequence SEQ ID No. 33 or a derivative thereof.

5. Polypeptide according to claim 3, characterized in that it is represented by the polypeptide sequence SEQ ID No. 22.

6. Nucleotide sequence encoding a polypeptide as defined according to one of claims 1 to 5.

7. Nucleotide sequence according to claim 6, characterized in that it comprises all or part of the sequence SEQ ID No. 15 or SEQ ID No. 21 or their derivatives.

8. Nucleotide sequence according to claim 6, characterized in that it comprises all or part of the nucleotide sequence SEQ ID No. 32 or its derivatives.

9. Nucleotide sequence according to claim 6 or 7, characterized in that it comprises the sequence SEQ ID No. 15 or the sequence SEQ ID No. 30.

10. Nucleotide sequence according to claim 6, 7 or 9, characterized in that it is represented in SEQ ID No. 21.

11. Host cell for the production of a polypeptide according to one of claims 1 to 5, characterized in that it was transformed with a nucleic acid containing a nucleotide sequence according to one of claims 6 to 10.

12. Method for preparing a polypeptide according to one of claims 1 to 5, characterized in that a cell containing a nucleotide sequence according to one of claims 6 to 10 is cultured under conditions for expressing said sequence, and the polypeptide produced is recovered.

13. Expression cassette comprising a nucleotide sequence encoding a polypeptide according to one of claims 6 to 10.

14. Vector comprising a nucleotide sequence according to one of claims 6 to 10.

15. Vector according to claim 14, characterized in that it is a plasmid vector, a cosmid or any DNA not encapsidated by a virus.

16. Vector according to claim 14, characterized in that it is a recombinant virus, and preferably a recombinant virus which is defective for replication.

17. Antisense oligonucleotide having a sequence according to claim 7 to 10 capable of at least partially inhibiting the production of polypeptides according to one of claims 1 to 5.

18. Nucleotide probe capable of hybridizing with a nucleotide sequence according to one of claims 7 to 10 or the corresponding mRNA.

19. Antibody or antibody fragment directed against a polypeptide according to one of claims 1 to 5.

20. Antibody or antibody fragment according to claim 19, characterized in that it is directed against a sequence chosen from the peptide sequences presented in SEQ ID No. 9 or SEQ ID No. 33 or SEQ ID No. 31 or SEQ ID No. 22.

21. Antibody or antibody fragment according to claim 19 or 20, characterized in that it possesses the ability to prevent the interaction between the oncogenic forms of p53 and a polypeptide according to one of claims 1 to 5.

22. Method for detecting or identifying compounds capable of binding with a polypeptide as defined according to one of claims 1 to 5, characterized in that the following steps are carried out:
a—a molecule or a mixture containing various molecules, optionally unidentified, is brought into contact with a polypeptide as defined according to one of claims 1 to 5 under conditions allowing interaction between said polypeptide and said molecule in the case where the latter might have affinity for said polypeptide, and,
b—the molecules bound to said polypeptide are detected and/or isolated.

23. Method for detecting or identifying compounds capable of modulating or inhibiting the interaction between between the oncogenic forms of p53 and a polypeptide according to one of claims 1 to 5, characterized in that the following steps are carried out:
a—the oncogenic form of p53 or a fragment thereof is bound to said polypeptide;
—a compound to be tested for its capacity to inhibit the binding between the oncogenic form of p53 and said polypeptide is added,
—the displacement or the inhibition of the binding between the oncogenic form of p53 is determined;
—the compounds which prevent or which impede the binding between the oncogenic form of p53 and said polypeptide are detected and/or isolated.

24. Ligand for a polypeptide as defined according to claims 1 to 5, which is capable of being obtained according to the method of claim 22.

25. Ligand capable of modulating or inhibiting the interaction between the oncogenic forms of p53 and a polypeptide as defined according to claims 1 to 5, which is capable of being obtained according to the method of claim 23.

26. Use of a ligand according to claim 24 or 25 for the preparation of a medicament intended for the treatment treatment of diseases involving a cell cycle dysfunction.

27. Use of a polypeptide capable of interacting with the oncogenic mutated forms of p53 and comprising all or part of a peptide sequence chosen from the sequences SEQ ID No. 9, No. 33, No. 31 or No. 22 or a derivative thereof according to one of claims 1 to 5 for the production of a nonpeptide or nonexclusively peptide compound capable of interacting with the oncogenic mutated forms of p53, by determining the structural components of this polypeptide which are important for its activity and reproducing these components by nonpeptide or nonexclusively peptide structures.

28. Pharmaceutical composition comprising, as active ingredient, at least one antibody or antibody fragment according to one of claims 19 to 21, and/or an antisense oligonucleotide according to claim 17 and/or a ligand according to either of claims 24 and 25 and/or a compound according to claim 27.

29. Composition according to claim 28 intended for the treatment of diseases involving a cell cycle dysfunction.

30. Composition according to claim 29 intended for the treatment of cancers.


SEQUENCE LISTING (33 sequences)

SEQ ID NO: 1 - DNA, 23 bp, Artificial Sequence
  Oligonucleotide
agatctgtatggaggagccgcag

SEQ ID NO: 2 - DNA, 29 bp, Artificial Sequence
  Oligonucleotide 3′ -393 (p53)
agatctcatcagtctgagtcaggcccttc

SEQ ID NO: 3 - DNA, 15 bp, Artificial Sequence
  Oligonucleotide H175 3′
ggggcagtgcctcac

SEQ ID NO: 4 - DNA, 15 bp, Artificial Sequence
  Oligonucleotide W248 3′
gggcctccagttcat

SEQ ID NO: 5 - DNA, 15 bp, Artificial Sequence
  Oligonucleotide H273 3′
acaaacatgcacctc

SEQ ID NO: 6 - DNA, 15 bp, Artificial Sequence
  Oligonucleotide G281 3′
gcgccggcctctccc

SEQ ID NO: 7 - DNA, 23 bp, Artificial Sequence
  Oligonucleotide 5′ -73
agatctgtgtggcccctgcacca

SEQ ID NO: 8 - DNA, 1021 bp, Artificial Sequence
  Fragment C-term MBP1 murine  CDS (1)...(885)
tgcacctgccctgatggttaccgaaaaattggacccgaatgtgtggacatagatgagtgtcgttaccgctattgccagcatcgatgtgtgaacctgccgggctcctttcgatgccagtgtgagccaggcttccagttgggacctaacaaccgctcttgtgtggatgtgaatgagtgtgacatgggagccccatgtgagcagcgctgcttcaactcctatgggaccttcctgtgtcgctgtaaccagggctatgagctgcaccgggatggcttctcctgcagcgatatcgatgagtgcggctactccagttacctctgccagtaccgctgtgtcaacgagccaggccgattctcctgtcactgcccacaaggctaccagctgctggctacaaggctctgccaagatattgacgagtgtgaaacaggtgcacaccaatgttctgaggcccaaacctgtgtcaacttccatgggggttaccgctgtgtggacaccaaccgttgtgtggagccctatgtccaagtgtcagacaaccgctgcctctgccctgcctccaatcccctttgtcgagagcagccttcatccattgtgcaccgctacatgagcatcacctcagagcgaagtgtgcctgctgacgtgtttcagatccaggcaacctctgtctaccctggtgcctacaatgcctttcagatccgttctggaaacacacagggggacttctacattaggcaaatcaacaatgtcagcgccatgctggtcctcgccaggccagtgacgggaccccgggagtacgtgctggacctggagatggtcaccatgaattcccttatgagctaccgggccagctctgtactgagactcacggtctttgtgggagcctataccttctgaagaccctcagggaagggccatgtgggggccccttccccctcccatagcttaagcagccccgggggcctagggatgaccgttctgcttaaaggaactatgatgtgaaggacaataaagggagaaagaaggaaaa

SEQ ID NO: 9 - protein, 295 aa, Artificial Sequence
  Fragment C-term MBP1 murine
CTCPDGYRKIGPECVDIDECRYRYCQHRCVNLPGSFRCQCEPGFQLGPNNRSCVDVNECDMGAPCEQRCFNSYGTFLCRCNQGYELHRDGFSCSDIDECGYSSYLCQYRCVNEPGRFSCHCPQGYQLLATRLCQDIDECETGAHQCSEAQTCVNFHGGYRCVDTNRCVEPYVQVSDNRCLCPASNPLCREQPSSIVHRYMSITSERSVPADVFQIQATSVYPGAYNAFQIRSGNTQGDFYIRQINNVSAMLVLARPVTGPREYVLDLEMVTMNSLMSYRASSVLRLTVFVGAYTF

SEQ ID NO: 10 - DNA, 39 bp, Artificial Sequence
  Oligonucleotide c-myc 5′
gatccatggagcagaagctgatctccgaggaggacctga

SEQ ID NO: 11 - DNA, 39 bp, Artificial Sequence
  Oligonucleotide c-myc 3′
gatctcaggtcctcctcggagatcagcttctgctccatg

SEQ ID NO: 12 - DNA, 45 bp, Artificial Sequence
  MCS 5′
gatctcggtcgacctgcatgcaattcccgggtgcggccgcgagct

SEQ ID NO: 13 - DNA, 37 bp, Artificial Sequence
  MCS 3′
cgcggccgcacccgggaattgcatgcaggtcgaccga

SEQ ID NO: 14 - DNA, 22 bp, Artificial Sequence
  Oligonucleotide 3′ mMBP1
cggtactggcagaggtaactgg

SEQ ID NO: 15 - DNA, 1513 bp, Artificial Sequence
  MBP1 murine (complete sequence) CDS (49)...(1377)
gctgtggcagaaacccctgacttctgcccaccacctcccagcctcaggatgctcccttttgcctcctgcctccccgggtctttgctgctctgggcgtttctgctgttgctcttgggagcagcgtccccacaggatcccgaggagccggacagctacacggaatgcacagatggctatgagtgggatgcagacagccagcactgccgggatgtcaacgagtgcctgaccatcccggaggcttgcaagggtgagatgaaatgcatcaaccactacgggggttatttgtgtctgcctcgctctgctgccgtcatcagtgatctccatggtgaaggacctccaccgccagcggcccatgctcaacaaccaaacccttgcccgcagggctacgagcctgatgaacaggagagctgtgtggatgtggacgagtgtacccaggctttgcatgactgtcgccctagtcaggactgccataaccttcctggctcctaccagtgcacctgccctgatggttaccgaaaaattggacccgaatgtgtggacatagatgagtgtcgttaccgctattgccagcatcgatgtgtgaacctgccgggctcttttcgatgccagtgtgagccaggcttccagttgggacctaacaaccgctcttgtgtggatgtgaatgagtgtgacatgggagccccatgtgagcagcgctgcttcaactcctatgggaccttcctgtgtcgctgtaaccagggctatgagctgcaccgggatggcttctcctgcagcgatatcgatgagtgcggctactccagttacctctgccagtaccgctgtgtcaacgagccaggccgattctcctgtcactgcccacaaggctaccagctgctggctacaaggctctgccaagatattgacgagtgtgaaacaggtgcacaccaatgttctgaggcccaaacctgtgtcaacttccatgggggttaccgctgtgtggacaccaaccgttgtgtggagccctatgtccaagtgtcagacaaccgctgcctctgccctgcctccaatcccctttgtcgagagcagccttcatccattgtgcaccgctacatgagcatcacctcagagcgaagtgtgcctgctgacgtgtttcagatccaggcaacctctgtctaccctggtgcctacaatgcctttcagatccgttctggaaacacacagggggacttctacattaggcaaatcaacaatgtcagcgccatgctggtcctcgccaggccagtgacgggaccccgggagtacgtgctggacctggagatggtcaccatgaattcccttatgagctaccgggccagctctgtactgagactcacggtctttgtgggagcctataccttctgaagaccctcagggaagggccatgtgggggccccttccccctcccatagcttaagcagccccgggggcctagggatgaccgttctgcttaaaggaactatgatgtgaaggacaataaagggagaaagaaggaaaa

SEQ ID NO: 16 - protein, 442 aa, Artificial Sequence
  MBP1 murine (complete sequence)
MLPFASCLPGSLLLWAFLLLLLGAASPQDPEEPDSYTECTDGYEWDADSQHCRDYNECLTIPEACKGEMKCINHYGGYLCLPRSAAVISDLHGEGPPPPAAHAQQPNPCPQGYEPDEQESCVDVDECTQALHDCRPSQDCHNLPGSYQCTCPDGYRKIGPECVDIDECRYRYCQHRCVNLPGSFRCQCEPGFQLGPNNRSCVDVNECDMGAPCEQRCFNSYGTFLCRCNQGYELHRDGFSCSDDECGYSSYLCQYRCVNEPGRFSCHCPQGYQLLATRLCQDIDECETGAHQCSEAQTCVNFHGGYRCVDTNRCVEPYVQVSDNRCLCPASNPLCREQPSSIVHRYMSITSERSVPADVFQIQATSVYPGAYNAFQIRSGNTQGDFYIRQINNVSAMLVLARPVTGPREYVLDLEMVTMNSLMSYRASSVLRLTVFVGAYTF

SEQ ID NO: 17 - DNA, 21 bp, Artificial Sequence
  Oligonucleotide 3′ hMBP1
ctccgctccgaggtgatggtc

SEQ ID NO: 18 - DNA, 21 bp, Artificial Sequence
  Oligonucleotide 5′ hMBP1
tgtagctactccagctacctc

SEQ ID NO: 19 - DNA, 1122 bp, Artificial Sequence
  Human cDNA MBP1
aagccagccgagccgccagagccgcgggccgcgggggtgtcgcgggcccaaccccaggatgctcccctgcgcctcctgcctacccgggtctctactgctctgggcgctgctactgttgctcttgggatcagcttctcctcaggattctgaagagcccgacagctacacggaatgcacagatggctatgagtgggacccagacagccagcactgccgggatgtcaacgagtgtctgaccatccctgaggcctgcaagggggaaatgaagtgcatcaaccactacgggggctacttgtgcctgccccgctccgctgccgtcatcaacgacctacacggcgagggacccccgccaccagtgcctcccgctcaacaccccaacccctgcccaccaggctatgagcccgacgatcaggacagctgtgtggatgtggacgagtgtgcccaggccctgcacgactgtcgccccagccaggactgccataacttgcctggctcctatcagtgcacctgccctgatggttaccgcaagatcgggcccgagtgtgtggacatagacgagtgccgctaccgctactgccagcaccgctgcgtgaacctgcctggctccttccgctgccagtgcgagccgggcttccagctggggcctaacaaccgctcctgtgttgatgtgaacgagtgtgacatgggggccccatgcgagcagcgctgcttcaactcctatgggaccttcctgtgtcgctgccaccagggctatgagctgcatcgggatggcttctcctgcagtgatattgatgagtgtagctactccagctacctctgtcagtaccgctgcgtcaacgagccaggccgtttctcctgccactgcccacagggttaccagctgctggccacacgcctctgccaagacattgatgagtgtgagtctggtgcgcaccagtgctccgaggcccaaacctgtgtcaacttccatgggggctaccgctgcgtggacaccaaccgctgcgtggagccctacatccaggtctctgagaaccgctgtctctgcccggcctccaaccctctatgtcgagagcagccttcatccattgtgcaccgctacatgaccatcacctcggagcggag

SEQ ID NO: 20 - DNA, 684 bp, Artificial Sequence
  Human cDNA MBP1 (partial sequence)
tgtagctactccagctacctctgtcagtaccgctgcgtcaacgagccaggccgtttctcctgccactgcccacagggttaccagctgctggccacacgcctctgccaagacattgatgagtgtgagtctggtgcgcaccagtgctccgaggcccaaacctgtgtcaacttccatgggggctaccgctgcgtggacaccaaccgctgcgtggagccctacatccaggtctctgagaaccgctgtctctgcccggcctccaaccctctatgtcgagagcagccttcatccattgtgcaccgctacatgaccatcacctcggagcggagcgtgcccgctgacgtgttccagatccaggcgacctccgtctaccccggtgcctacaatgcctttcagatccgtgctggaaactcgcagggggacttttacattaggcaaatcaacaacgtcagcgccatgctggtcctcgcccggccggtgacgggcccccgggagtacgtgctggacctggagatggtcaccatgaattccctcatgagctaccgggccagctctgtactgaggctcaccgtctttgtaggggcctacaccttctgaggagcaggagggagccaccctccctgcagctaccctagctgaggagcctgttgtgaggggcagaatgagaaaggcaataaagggagaaag

SEQ ID NO: 21 - DNA, 1422 bp, Artificial Sequence
  Human MBP1 (complete sequence) CDS (59)... (1387)
atgctcccctgcgcctcctgcctacccgggtctctactgctctgggcgctgctactgttgctcttgggatcagcttctcctcaggattctgaagagcccgacagctacacggaatgcacagatggctatgagtgggacccagacagccagcactgccgggatgtcaacgagtgtctgaccatccctgaggcctgcaagggggaaatgaagtgcatcaaccactacgggggctacttgtgcctgccccgctccgctgccgtcatcaacgacctacacggcgagggacccccgccaccagtgcctcccgctcaacaccccaacccctgcccaccaggctatgagcccgacgatcaggacagctgtgtggatgtggacgagtgtgcccaggccctgcacgactgtcgccccagccaggactgccataacttgcctggctcctatcagtgcacctgccctgatggttaccgcaagatcgggcccgagtgtgtggacatagacgagtgccgctaccgctactgccagcaccgctgcgtgaacctgcctggctccttccgctgccagtgcgagccgggcttccagctggggcctaacaaccgctcctgtgttgatgtgaacgagtgtgacatgggggccccatgcgagcagcgctgcttcaactcctatgggaccttcctgtgtcgctgccaccagggctatgagctgcatcgggatggcttctcctgcagtgatattgatgagtgtagctactccagctacctctgtcagtaccgctgcgtcaacgagccaggccgtttctcctgccactgcccacagggttaccagctgctggccacacgcctctgccaagacattgatgagtgtgagtctggtgcgcaccagtgctccgaggcccaaacctgtgtcaacttccatgggggctaccgctgcgtggacaccaaccgctgcgtggagccctacatccaggtctctgagaaccgctgtctctgcccggcctccaaccctctatgtcgagagcagccttcatccattgtgcaccgctacatgaccatcacctcggagcggagcgtgcccgctgacgtgttccagatccaggcgacctccgtctaccccggtgcctacaatgcctttcagatccgtgctggaaactcgcagggggacttttacattaggcaaatcaacaacgtcagcgccatgctggtcctcgcccggccggtgacgggcccccgggagtacgtgctggacctggagatggtcaccatgaattccctcatgagctaccgggccagctctgtactgaggctcaccgtctttgtaggggcctacaccttctgaggagcaggagggagccaccctccctgcagctaccctagctgaggagcctgttgtgaggggcagaatgagaaaggcaataaagggagaaag

SEQ ID NO: 22 - protein, 443 aa, Artificial Sequence
  Human MBP1 (complete sequence)
MLPCASCLPGSLLLWALLLLLLGSASPQDSEEPDSYTECTDGYEWDPDSQHCRDVNECLTIPEACKGEMKCINHYGGYLCLPRSAAVINDLHGEGPPPPVPPAQHPNPCPPGYEPDDQDSCVDVDECAQALHDCRPSQDCHNLPGSYQCTCPDGYRKIGPECVDIDECRYRYCQHRCVNLPGSFRCQCEPGFQLGPNNRSCVDVNECDMGAPCEQRCFNSYGTFLCRCHQGYELHRDGFSCSDIDECSYSSYLCQYRCVNEPGRFSCHCPQGYQLLATRLCQDIDECESGAHQCSEAQTCVNFHGGYRCVDTNRCVEPYIQVSENRCLCPASNPLCREQPSSIVHRYMTITSERSVPADVFQIQATSVYPGAYNAFQIRAGNSQGDFYIRQINNVFAMLVLARPVTGPREYVLDLEMVTMNSLMSYRASSVLRLTVFVGAYTF

SEQ ID NO: 23 - DNA, 817 bp, Artificial Sequence
  cDNA MBP1 murine (partial sequence)
gctgtggcagaaacccctgacttctgcccaccacctcccagcctcaggatgctcccttttgcctcctgcctccccgggtctttgctgctctgggcgtttctgctgttgctcttgggagcagcgtccccacaggatcccgaggagccggacagctacacggaatgcacagatggctatgagtgggatgcagacagccagcactgccgggatgtcaacgagtgcctgaccatcccggaggcttgcaagggtgagatgaaatgcatcaaccactacgggggttatttgtgtctgcctcgctctgctgccgtcatcagtgatctccatggtgaaggacctccaccgccagcggcccatgctcaacaaccaaacccttgcccgcagggctacgagcctgatgaacaggagagctgtgtggatgtggacgagtgtacccaggctttgcatgactgtcgccctagtcaggactgccataaccttcctggctcctaccagtgcacctgccctgatggttaccgaaaaattggacccgaatgtgtggacatagatgagtgtcgttaccgctattgccagcatcgatgtgtgaacctgccgggctcttttcgatgccagtgtgagccaggcttccagttgggacctaacaaccgctcttgtgtggatgtgaatgagtgtgacatgggagccccatgtgagcagcgctgcttcaactcctatgggaccttcctgtgtcgctgtaaccagggctatgagctgcaccgggatggcttctcctgcagcgatatcgatgagtgcggctactccagttacctctgccagtacc

SEQ ID NO: 24 - DNA, 24 bp, Artificial Sequence
  Oligonucleotide sens-GAPDH
cggagtcaacggatttggtcgtat

SEQ ID NO: 25 - DNA, 24 bp, Artificial Sequence
  Oligonucleotide antisens-GAPDH
agccttctccatggtggtgaagac

SEQ ID NO: 26 - DNA, 25 bp, Artificial Sequence
  Oligonucleotide
cggttggccttggggttcagggggg

SEQ ID NO: 27 - DNA, 21 bp, Artificial Sequence
  Oligonucleotide sens MBP1
gccctgatggttaccgcaaga

SEQ ID NO: 28 - DNA, 21 bp, Artificial Sequence
  Oligonucleotide antisens MBP1
agcccccatggaagttgacac

SEQ ID NO: 29 - DNA, 20 bp, Artificial Sequence
  Oligonucleotide sens actine
gtggggcgccccaggcacca

SEQ ID NO: 30 - DNA, 1358 bp, Artificial Sequence
  Human fragment C-term MBP1  CDS (1)...(885)
tgcacctgccctgatggttaccgcaagatcgggcccgagtgtgtggacatagacgagtgccgctaccgctactgccagcaccgctgcgtgaacctgcctggctccttccgctgccagtgcgagccgggcttccagctggggcctaacaaccgctcctgtgttgatgtgaacgagtgtgacatgggggccccatgcgagcagcgctgcttcaactcctatgggaccttcctgtgtcgctgccaccagggctatgagctgcatcgggatggcttctcctgcagtgatattgatgagtgtagctactccagctacctctgtcagtaccgctgcgtcaacgagccaggccgtttctcctgccactgcccacagggttaccagctgctggccacacgcctctgccaagacattgatgagtgtgagtctggtgcgcaccagtgctccgaggcccaaacctgtgtcaacttccatgggggctaccgctgcgtggacaccaaccgctgcgtggagccctacatccaggtctctgagaaccgctgtctctgcccggcctccaaccctctatgtcgagagcagccttcatccattgtgcaccgctacatgaccatcacctcggagcggagcgtgcccgctgacgtgttccagatccaggcgacctccgtctaccccggtgcctacaatgcctttcagatccgtgctggaaactcgcagggggacttttacattaggcaaatcaacaacgtcagcgccatgctggtcctcgcccggccggtgacgggcccccgggagtacgtgctggacctggagatggtcaccatgaattccctcatgagctaccgggccagctctgtactgaggctcaccgtctttgtaggggcctacaccttctgaggagcaggagggagccaccctccctgcagctaccctagctgaggagcctgttgtgaggggcagaatgagaaaggcaataaagggagaaagaaagtcctggtggctgaggtgggcgggtcacactgcaggaagcctcaggctggggcagggtggcacttgggggggcaggccaagttcacctaaatgggggtctctatatgttcaggcccaggggcccccattgacaggagctgggagctctgcaccacgagcttcagtcaccccgagaggagaggaggtaacgaggagggcggactccaggccccggcccagagatttggacttggctggcttgcaggggtcctaagaaactccactctggacagcgccaggaggccctgggttccattcctaactctgcctcaaactgtacatttggataagccctagtagttccctgggcctgtttttctataaaacgaggcaactgg

SEQ ID NO: 31 - protein, 295 aa, Artificial Sequence
  Human fragment C-term MBP1
CTCPDGYRKIGPECVDIDECRYRYCQHRCVNLPGSFRCQCEPGFQLGPNNRSCVDVNECDMGAPCEQRCFNSYGTFLCRCHQGYELHRDGFSCSDIDECSYSSYLCQYRCVNEPGRFSCHCPQGYQLLATRLCQDIDECESGAHQCSEAQTCVNFHGGYRCVDTNRCVEPYIQVSENRCLCPASNPLCREQPSSIVHRYMTITSERSVPADVFQIQATSVYPGAYNAFQIRAGNSQGDFYIRQINNVSAMLVLARPVTGPREYVLDLEMVTMNSLMSYRASSVLRLTVFVGAYTF

SEQ ID NO: 32 - DNA, 1663 bp, Artificial Sequence
  Fragment c-term fibuline 2 murine  CDS (1)... (999)
gagggctctgaatgtgtggatgtgaatgagtgtgagacaggtgtgcatcgctgtggcgagggccaactgtgctataacctccctggatcctaccgctgtgactgcaagcccggcttccagagggatgcattcggcaggacttgcattgatgtgaacgaatgctgggtctcgccgggccgcctgtgccagcacacatgtgagaacacaccgggctcctaccgctgctcctgcgctgctggcttccttttggccgcagatggcaaacattgtgaagatgtgaacgagtgcgagactcggcgctgcagccaggaatgtgccaacatctatggctcctatcagtgctactgccgtcagggctaccagctggcagaggatgggcatacctgcacagacatcgatgagtgtgcacagggcgcgggcattctctgtaccttccgctgtgtcaacgtgcctgggagctaccagtgtgcatgcccagagcaagggtatacaatgatggccaacgggaggtcctgcaaggacctggatgagtgtgcactgggcacccacaactgctctgaggctgagacctgccacaatatccaggggagtttccgctgcctgcgctttgattgtccacccaactatgtccgtgtctcacaaacgaagtgcgagcgcaccacatgccaggatatcacggaatgtcaaacctcaccagctcgcatcacgcactaccagctcaatttccagacaggcctactggtacctgcacatatcttccgcatcggccctgctcccgcctttgctggggacaccatctccctgaccatcacgaagggcaatgaggagggctacttcgtcacacgcagactcaatgcctacactggtgtggtatccctgcagcggtctgttctggagccgcgggactttgccctagatgtggagatgaagctttggcgccagggctctgtcactaccttcctggccaagatgtacatcttcttcaccacttttgccccatgaggtgacatgtcaggcaatccctccaggtgatgcctgggcggtgggcagctgcgccactcctaagtggctttttgctgtgactctgtaacttaacttaatcatgctgagctggttggtcttgagtctctaccctagagggagggagatgcaccccagcaggcactgagtacaggccagggtcacccgaggctagatggtgacctgcaaactggaaacagccatagggggcttctgaactccactcctcaactatggctacagctgacattccattccttcatccactgtgttcctcaattaaaaaaaaaaatcagctgtgcatggtagcacagacctttaatcctagcactggggaggcagaggtaggtagatctctgagttccaggccagcctggtctacactgggagttctaaccagccagagctacatagagagaccctatctcaacaaggaaaaaacgaaagaaatctctgtgagttccaggccagcctggtctacgctgggagttctaaccagccagagctacatagagagatcctatctcaacaaggaaaaatgaaagaaatcattttaaaaggtttttttttttgctgttgttgtttaatgataagagtagcacatatacattattaaaaatgatcaaatagcacagaaaggtta

SEQ ID NO: 33 - protein, 333 aa, Artificial Sequence
  Fragment c-term fibuline 2 murine
EGSECVDVNECETGVHRCGEGQLCYNLPGSYRCDCKPGFQRDAFGRTCIDVNECWVSPGRLCQHTCENTPGSYRCSCAAGFLLAADGKHCEDVNECETRRCSQECANIYGSYQCYCRQGYQLAEDGHTCTDIDECAQGAGILCTFRCVNVPGSYQCACPEQGYTMMANGRSCKDLDECALGTHNCSEAETCHNIQGSFRCLRFDCPPNYVRVSQTKCERTTCQDITECQTSPARITHYQLNFQTGLLVPAHIFRIGPAPAFAGDTISLTITKGNEEGYFVTRRLNAYTGVVSLQRSVLEPRDFALDVEMKLWRQGSVTTFLAKMYIFFTTFAP